(19) United States
(12) Patent Application Publication
(10) Pub. No.: US 2025/0359736 A1
(43) Pub. Date: Nov. 27, 2025

(54) METHODS AND APPARATUSES FOR NAVIGATING USING A PAIR OF RIGIDIZING DEVICES

(71) Applicant: Neptune Medical Inc., Burlingame, CA (US)

(72) Inventors: Natalie FERRANTE, San Francisco, CA (US); Neal TANNER, Austin, TX (US)

(21) Appl. No.: 18/851,053

(22) PCT Filed: Mar. 27, 2023
(86) PCT No.: PCT/US2023/064999

§ 371 Date: Sep. 25, 2024

Related U.S. Application Data
(60) Provisional application No. 63/324,011, filed on Mar. 25, 2022.

(51) Int. Cl. A61B 1/005; A61B 1/00

(52) U.S. Cl. CPC A61B 1/0055; A61B 1/00006; A61B 1/00135

(57) ABSTRACT

Disclosed herein are systems and methods for navigating a system comprising a nested pair of rigidizing devices through a body lumen. The rigidizing devices are configured to alternately rigidize and to transition between a rigidized state and a flexible state and to copy the shape of the device when advancing or retracting. Also described herein are examples of apparatuses for automatically or semi-automatically screening a body lumen using a nested pair of rigidizing devices.

### PRIORITY CLAIM

This patent application claims priority to 63/324,011, filed Mar. 25, 2022, titled “METHODS AND APPARATUSES FOR NAVIGATING USING A PAIR OF RIGIDIZING DEVICES,” herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

During medical procedures, a nested or telescoping apparatus may advance by the coordinated movement of both an inner device (inner member, child, catheter, endoscope, etc.) and an outer device (e.g., outer member, mother, overtube, etc.). However, it may be particularly challenging to coordinate the movement of the inner and outer members when navigating through curving or tortuous anatomy, including body regions in which the device may bend, curve and even loop or double over itself. This may make both advancement and/or retraction of the medical device difficult.

These problems may be particularly acute in anatomical regions such as the gastrointestinal region that may be tortuous and may form loops. Gastrointestinal looping, caused when a traditional endoscope can no longer advance due to excessive curving or looping of the gastrointestinal tract, is a particularly well-known clinical challenge for endoscopy. Gastrointestinal looping prolongs the procedure and can cause pain to the patient because it can stretch the vessel wall and the mesentery. Furthermore, gastrointestinal looping leads to an increased incidence of perforations. Similar problems commonly occur across a wide range of endoscopic procedures, including colonoscopy, esophagogastroduodenoscopy (EGD), enteroscopy, endoscopic retrograde cholangiopancreatography (ERCP), interventional endoscopy procedures (including ESD (Endoscopic Submucosal Dissection) and EMR (Endoscopic Mucosal Resection)), robotic flexible endoscopy, trans-oral robotic surgery (TORS), altered anatomy cases (including Roux-en-Y), and during NOTES (Natural Orifice Transluminal Endoscopic Surgery) procedures. Accordingly, there is a need for apparatuses that may prevent gastrointestinal looping and/or may otherwise provide more successful access to the gastrointestinal tract. In particular, there is a need for methods and apparatuses that can safely and efficiently coordinate the movement of all telescoping members of a nested apparatus having both an inner and outer rigidizing member during advancement and retraction of the apparatuses.

### SUMMARY OF THE DISCLOSURE

Described herein are methods and apparatuses (e.g., devices, systems, etc.) for controlling a pair of nested and selectably rigidizing members during advancement and retraction within a body lumen. One or both of the selectably rigidizable members may be steerable at a distal end region of the member by applying tenson to one or more tendons to cause bending of the distal end region in a desired direction. In some examples an apparatus may include a pair of nested rigidizing devices that can be alternately rigidized and advanced (or retracted) distally or proximally through a body lumen. In the absence of constraints, the transition between the rigidized and flexible states can involve a significant shape change, which can, in some circumstances, be harmful to surrounding anatomy. There is a need for methods and systems that can provide safe and smooth transitions between the rigidized and flexible states for such devices.

In general, these methods and apparatuses may include estimating the current shape of the nested set by remembering the sequence of commanded articulations and copies, and/or using the estimated shape to improve control of the guidance (e.g., steering) of the apparatuses within space, such as within a body lumen. The memory of the current shape may be recalled (“remembered”) by using a first, flexible, device to copy a shape of a second, rigidized (e.g., locked), device that is nested with the first device.

In some examples these methods and apparatuses may be used to withdraw (proximally) the apparatus including the pair of nested devices, while maintaining the orientation of the distal tip of the apparatus, e.g., so that a camera on a distal face of the first or second rigidizing device may maintain its orientation (e.g., maintaining a net articulation angle). This may advantageously allow these apparatuses to smoothly transition between rigid and non-rigid states without substantially deflecting. For example, a method of controlling a nested pair of rigidizing devices, the method comprising: retracting a first rigidizing device of the nested pair of rigidizing devices relative to a second rigidizing device of the nested pair of rigidizing devices, while the first rigidizing device is in a flexible state and the second rigidizing device is in a rigidized state; rigidizing the first rigidizing device; and actuating steering members within the second rigidizing device to maintain a direction of a distal end face of the second rigidizing device constant relative to an external region before and/or while transitioning the second rigidizing device from the rigid state to the flexible state.

Any of these methods or apparatuses may include, after actuating the steering members, retracting the second rigidizing device relative to the first rigidizing device while the second rigidizing device is in the flexible state. These methods and apparatuses may further be configured to rigidize the second rigidizing device and retract the first rigidizing device over the second rigidizing device while the first rigidizing device is in a flexible state. Retracting the second rigidizing device may comprise retracting the second rigidizing device at least partially into the first rigidizing device. In any of these methods and apparatuses (e.g., systems) actuating the steering members within the second rigidizing device to maintain the direction of the distal end face of the second rigidizing device may comprise maintaining a net articulation angle between a distal end of the second rigidizing device with respect to a proximal portion of the second rigidizing device. In any of these methods and apparatuses, actuating the steering members within the second rigidizing device to maintain the direction of the distal end face of the second rigidizing device constant relative to the external region may comprise maintaining the direction of the distal end face of the second rigidizing device relative to a lumen wall. For example, actuating the steering members within the second rigidizing device to maintain the direction of the distal end face of the second rigidizing device constant relative to the external region comprises maintaining the direction of the distal end face of the second rigidizing device so that the distal end face of the second rigidizing device varies less than +/−15 degrees of angular direction. Actuating the steering members may comprise applying tension to at least one of the steering members. In some examples actuating the steering members comprises displacing at least one of the steering members. Any of these methods and apparatuses may include actuating the steering members by automatically actuating the steering members.

The methods and apparatuses described herein may include retracting the first rigidizing device comprises retracting the first rigidizing device proximally relative to the second rigidizing device.

The first rigidizing device may be nested within the second rigidizing device. Alternatively, the second rigidizing device may be nested within the first rigidizing device.

The methods and apparatuses described herein may include imaging the external region from a sensor at the distal end face of the second rigidizing device.

Also described herein are apparatuses (e.g., systems) configured to perform any of the methods described herein. These apparatuses may include a nested pair of rigidizing devices comprising a first rigidizing device and a second rigidizing device; one or more processors; and a memory coupled to the one or more processors, the memory storing computer-program instructions, that, when executed by the one or more processors, perform the computer-implemented method for controlling the nested pair of rigidizing devices.

Also described herein are methods for automatically (or semi-automatically) shape copying. In particular, described herein are methods of automatically shape copying when triggered by a user actuating a control, and continuing the automatic shape-copying while the user continues to actuating the control, but stopping if the user stops actuating the control. For example, the control may be actuated by pressing a button or switch; the shape-copying procedure may be automatically performed while the user presses the button but stops when the user stops pressing the button.

For example, a method of controlling a nested pair of rigidizing devices may include: receiving a copy command from a user input; automatically performing a shape copying sequence, wherein the shape copying sequence comprises: advancing a first rigidizing device of the nested pair of rigidizing devices relative to a second rigidizing device of the nested pair of rigidizing devices, while the first rigidizing device is in a flexible state and the second rigidizing device is in a rigidized state, wherein the first rigidizing device is initially proximal to the second rigidizing device so that first rigidizing device copies the shape of the second rigidizing device; and preventing the first rigidizing device from advancing distal to the second rigidizing device.

Any of these methods may include continuing advancement of the first rigidizing device until the distal end of the first rigidizing device reaches the distal end of the second rigidizing device.

As mentioned, advancing the first rigidizing device may comprise advancing the first rigidizing device only while the copy command is continuously received. Any of these methods (or apparatuses) may further or alternatively be configured to prevent the first device (e.g., the first rigidizing device) beyond the distal end region of the second device (e.g., the second rigidizing device). These feature allow for rapid and efficient operation of these nested systems, while maintaining a high degree of user control that is not possible without preventing overshoot and/or stopping partially through an automatic shape copying procedure.

The shape copying sequence may include rigidizing the second rigidizing device into the rigid state prior to advancing the first rigidizing device. In some examples the shape copying sequence further comprises de-rigidizing the first rigidizing device into the flexible state prior to advancing the first rigidizing device relative to the second rigidizing device. The shape copying sequence may further comprise rigidizing the first rigidizing device into the rigid state after it has advanced relative to the second rigidizing device. In any of these method and apparatuses, prior to receiving the copy command, the method may include advancing the second rigidizing device in the flexible state while steering a distal end region of the second rigidizing device, wherein the first rigidizing device is in the rigid state.

For example, a method of controlling a nested pair of rigidizing devices may include: receiving a copy command from a user input; and automatically performing a shape copying sequence while the user input is received, wherein the shape copying sequence comprises: advancing a first rigidizing device of the nested pair of rigidizing devices relative to the first to a second rigidizing device of the nested pair of rigidizing devices, while the first rigidizing device is in a flexible state and the second rigidizing device is in a rigidized state, wherein the first rigidizing device is initially proximal to the second rigidizing device so that first rigidizing device copies the shape of the second rigidizing device.

Also described herein are methods and apparatuses (e.g., systems) for automatically shape copying when detecting an automatic copying trigger. The automatic copying trigger may be, e.g., extending one of the first or second nested rigidizing devices to far distally relative to the other rigidizing device, a time delay of longer than a set threshold when moving one of the nested rigidizing devices relative to the other, a lag in the movement of one or the other rigidizing devices, etc.

For example, a method of controlling a nested pair of rigidizing devices including a first rigidizing device and a second rigidizing device, the method comprising automatically performing a shape copying sequence when an automatic copying trigger event is detected by a control circuitry, the method comprising: receiving, in the controller, one or more of sensor data and/or user movement input; comparing the one or more of sensor data and/or user movement input to an automatic copying trigger threshold; and triggering the shape copying sequence when the automatic copying trigger threshold is detected, wherein the shape copying sequence comprises: advancing the first rigidizing device relative to the second rigidizing device, while the first rigidizing device is in a flexible state and the second rigidizing device is in a rigidized state.

As mentioned, in some examples the automatic copying trigger threshold may be a relative axial travel distance between the first rigidizing member and the second rigidizing member. The automatic copying trigger threshold may comprise exceeding a time delay following movement of the second rigidizing member relative to the first rigidizing member. Automatically performing the shape copying sequence may comprise advancing the first rigidizing device relative to the second rigidizing device until a distal end region of the first rigidizing device is adjacent to a distal end region of the second rigidizing device.

The first rigidizing device may be nested over the second rigidizing device.

The shape copying sequence may further comprise comprising rigidizing the second rigidizing device into the rigid state prior to advancing the first rigidizing device. In some examples the shape copying sequence further comprises de-rigidizing the first rigidizing device into the flexible state prior to advancing the first rigidizing device relative to the second rigidizing device. The shape copying sequence may further comprise rigidizing the first rigidizing device into the rigid state after it has advanced relative to the second rigidizing device.

A method of controlling a nested pair of rigidizing devices may include: advancing a second rigidizing device of the nested pair of rigidizing devices distally relative to a first rigidizing device of the nested pair of rigidizing devices, while the second rigidizing device is in a flexible state and the first rigidizing device is in a rigidized state; and automatically performing a shape copying sequence when the second rigidizing member extends to a predetermined travel distance relative to the first rigidizing member, wherein the shape copying sequence comprises: advancing the first rigidizing device relative to the second rigidizing device, while the first rigidizing device is in a flexible state and the second rigidizing device is in a rigidized state.

The predetermined travel distance may be a maximum distance. Advancing the second rigidizing device may comprise advancing and steering.

As mentioned, automatically performing the shape copying sequence may comprise advancing the first rigidizing device relative to the second rigidizing device until a distal end region of the first rigidizing device is adjacent to a distal end region of the second rigidizing device. The shape copying sequence may further comprise comprising rigidizing the second rigidizing device into the rigid state prior to advancing the first rigidizing device. The shape copying sequence may further comprise de-rigidizing the first rigidizing device into the flexible state prior to advancing the first rigidizing device relative to the second rigidizing device. The shape copying sequence may further comprise rigidizing the first rigidizing device into the rigid state after it has advanced relative to the second rigidizing device.

Any of the methods and apparatuses described herein may also include controlling the timing of the release of tension of the actuating steering members of the steerable distal end of a nested rigidizing device when transitioning from a rigid to a flexible state, e.g., by actuating steering members to maintain a curvature as the device transitions to the flexible state.

For example, a method of controlling a nested pair of rigidizing devices may include: advancing a first rigidizing device of the nested pair of rigidizing devices distally relative to a second rigidizing device of the nested pair of rigidizing devices, wherein the first rigidizing device is in a flexible state and the second rigidizing device is in a rigid state; transitioning the first rigidizing device from the flexible state to the rigid state; and transitioning the second rigidizing device from the rigid state to the flexible state while actuating steering members of the second rigidizing device to maintain a curvature of a distal end of the second rigidizing device as the second rigidizing device transitions to the flexible state.

Any of these methods may include advancing the flexible second rigidizing device distally and further actuating the steering members to steer the distal end of the second rigidizing device while the first rigidizing device remains in the rigid state. Actuating the steering members within the second rigidizing device may include pulling on one or more tendons. Actuating the steering members may comprise applying tension to at least one of the steering members. Actuating the steering members may comprise automatically actuating the steering members.

In some examples the first rigidizing device is nested within the second rigidizing device. Any of these methods may include imaging the external region from a sensor at the distal end face of the second rigidizing device, and/or advancing the second rigidizing device in the flexible state and steering the second rigidizing device while advancing, then repeating the steps of advancing a first rigidizing device distally, transitioning the first rigidizing device to the rigid state and transitioning the second rigidizing device to the flexible state.

Also described herein are methods of advancing or retracting a system comprising a nested pair of rigidizing devices along a body lumen. Any of these methods may include: advancing or retracting a first rigidizing device in a flexible state relative to a second rigidized device in a rigid state and steering the first rigidizing device using steering members coupled to the first rigidizing device; rigidizing the first rigidizing device; advancing or retracting the second rigidizing device in a flexible state at least partially over the rigidized first rigidizing device; rigidizing the second rigidizing device; actuating the steering members to correspond to a curvature of the rigidized second rigidizing device; and transitioning the first rigidizing device to a flexible state.

Actuating the steering members in the first rigidizing device may comprise maintaining the previously commanded curvature of the first rigidizing device throughout the advancing or retracting and rigidizing of the second rigidizing device. In some examples, actuating the steering members in the first rigidizing device comprises adjusting the steering members to correspond to a new curvature of the rigidized second rigidizing device that is different from the curvature prior to rigidizing the first rigidizing device before transitioning the first rigidizing device to a flexible state. In some examples actuating the steering members is performed prior to transitioning the first rigidizing device to a flexible state. Actuating the steering members may be performed while transitioning the first rigidizing device to a flexible state.

In any of these methods, the system may be configured to automatically maintain existing curvature command controls on the steering members during advancing or retracting and rigidizing of the second rigidizing device.

Advancing or retracting the second rigidizing device may comprise advancing the second rigidizing device such that a distal end of the second rigidizing device is generally aligned with a distal end of the first rigidizing device. In some examples the system may be configured to automatically maintain existing curvature command controls on the steering members during advancing or retracting and rigidizing of the second rigidizing device.

The system may be configured to automatically actuate the steering members while the first rigidizing device transitions to the flexible state. Actuating the steering members may comprise applying tension to at least one of the steering members. Actuating the steering members may comprise displacing at least one of the steering members. The system may be configured to automatically actuate the steering members to impart a curvature to the first rigidizing device that is a predetermined percentage less than the previously commanded curvature imposed by the steering members.

Advancing or retracting the first and second rigidizing devices may comprise advancing distally. Advancing or retracting the first and second rigidizing devices may comprise retracting proximally.

In some examples, actuating the steering members may comprises actuating the steering members to correspond to a shape of a portion of the first rigidizing device to be exposed upon proximally retracting the second rigidizing device over the first rigidizing device. Actuating the steering members may comprise approximating an angle between a distal face of the first rigidizing device and a cross section of the first rigidizing device at a proximal end of the portion of the first rigidizing device. For example, actuating the steering members may comprise maintaining an orientation of a distal end of the first rigidizing device with respect to a proximal end of the portion of the first rigidizing device.

For example, a method of advancing a system comprising a nested pair of rigidizing devices along a body lumen may include: advancing a first rigidizing device in a flexible state through the body lumen and steering a distal end region of the first rigidizing device using steering members coupled to the first rigidizing device; rigidizing the first rigidizing device; advancing a second rigidizing device in a flexible state at least partially over the rigidized first rigidizing device; rigidizing the second rigidizing device; actuating the steering members to correspond to a new curvature of the rigidized second rigidizing device that is different from the curvature prior to rigidizing the first rigidizing device before transitioning the first rigidizing device to a flexible state; and transitioning the first rigidizing device to the flexible state.

A method of retracting a system comprising a nested pair of rigidizing devices along a body lumen may include: retracting a first rigidizing device in a flexible state through the body lumen relative to a second rigidizing device in a rigidized state and steering a distal end region of the first rigidizing device using steering members coupled to the first rigidizing device; rigidizing the first rigidizing device; retracting the second rigidizing device in a flexible state at least partially over the rigidized first rigidizing device; rigidizing the second rigidizing device; actuating the steering members to maintain an orientation of a distal end of the first rigidizing device with respect to a proximal portion of the second rigidizing device; and retracting the first rigidizing device into the second rigidizing device while the first rigidizing device is in a flexible state.

For example, a method of retracting a system comprising a nested pair of rigidizing devices along a body lumen may comprise: retracting a first rigidizing device in a flexible state through the body lumen relative to a second rigidizing device in a rigidized state and steering a distal end region of the first rigidizing device using steering members coupled to the first rigidizing device; rigidizing the first rigidizing device; retracting the second rigidizing device in a flexible state at least partially over the rigidized first rigidizing device; rigidizing the second rigidizing device; actuating the steering members to maintain a constant curvature; and retracting the first rigidizing device into the second rigidizing device while the first rigidizing device is in a flexible state.

Also described herein are systems comprising: a first rigidizing device positioned within a second rigidizing device; a controller comprising one or more processors; and a memory coupled to the one or more processors, the memory storing computer-program instructions, that, when executed by the one or more processors, perform a computer-implemented method to advance the inner and outer rigidizing devices within a body lumen, the method comprising: advancing or retracting a first rigidizing device in a flexible state relative to a second rigidized device in a rigid state and steering the first rigidizing device using steering members coupled to the first rigidizing device; rigidizing the first rigidizing device; advancing or retracting the second rigidizing device in a flexible state at least partially over the rigidized first rigidizing device; rigidizing the second rigidizing device; actuating the steering members to correspond to a curvature of the rigidized second rigidizing device; and transitioning the first rigidizing device to a flexible state.

In any of these apparatuses and methods the inner rigidizing device may be referred to as an inner member, child, catheter, endoscope, etc., and an outer rigidizing device may be referred to as an outer member, mother, overtube, etc. The outer rigidizing device may be referred to as a first rigidizing device and the inner rigidizing device may be referred to as the second rigidizing device; alternatively, the outer rigidizing device may be referred to as the second rigidizing device and the inner rigidizing device may be referred to as the first rigidizing device. In general, the inner rigidizing device is nested within the outer rigidizing device and the outer rigidizing device is nested over the inner rigidizing device, so that the two rigidizing devices may move longitudinally (distally and proximally) with respect to each other.

In any of these examples the first (inner) rigidizing device may retract into a non-straight (e.g., curved, bent, etc.) distal section of the second (outer) rigidizing member, and may compensate for that non-strait region by articulating the bending section to match that curve, for example when the first rigidizing device pulls back into the second rigidizing device.

In any of these apparatuses, actuating the steering members in the first rigidizing device may comprise maintaining the previously commanded curvature of the first rigidizing device throughout the advancing and rigidizing of the second rigidizing device. In some examples actuating the steering members in the first rigidizing device may comprise adjusting the steering members to correspond to a shape of the first rigidizing device to the rigidized shape of the second rigidizing device. Actuating the steering members may be performed prior to transitioning the first rigidizing device to a flexible state. Actuating the steering members may be performed while transitioning the first rigidizing device to a flexible state.

Any of these systems may be configured to automatically maintain existing curvature command controls on the steering members during advancing and rigidizing of the second rigidizing device.

In some examples advancing the second rigidizing device may comprise advancing the second rigidizing device such that a distal end of the second rigidizing device is generally aligned with a distal end of the first rigidizing device. The system may be configured to automatically maintain existing curvature command controls on the steering members during advancing and rigidizing of the second rigidizing device. In some examples the system is configured to automatically actuate the steering members while the first rigidizing device transitions to the flexible state.

In any of these examples actuating the steering members comprises applying tension to at least one of the steering members. For example, actuating the steering members may comprise displacing at least one of the steering members. The system may be configured to automatically actuate the steering members to impart a curvature to the first rigidizing device that is a predetermined percentage less than the previously commanded curvature imposed by the steering members. In some examples, advancing the first and second rigidizing devices comprises advancing distally. Advancing the first and second rigidizing devices may comprise advancing proximally.

Actuating the steering members may comprise actuating the steering members to correspond to a shape of a portion of the first rigidizing device to be exposed upon proximally retracting the second rigidizing device over the first rigidizing device. Actuating the steering members may comprise approximating an angle between a distal face of the first rigidizing device and a cross section of the first rigidizing device at a proximal end of the portion of the first rigidizing device. In some examples actuating the steering members comprises maintaining a position of a distal end of the first rigidizing device with respect to a proximal end of the portion of the first rigidizing device.

Also described herein are methods of screening a body lumen of a patient, the method comprising: navigating a system comprising a first rigidizing device positioned within a second rigidizing device through the body lumen, the first rigidizing device comprising a camera at a distal end; exposing a distal portion of the first rigidizing device; articulating the distal portion of the first rigidizing device to perform a circular pass (e.g., perform a loop motion with the tip) resulting in visualization by the camera of a circumference of a first portion of the body lumen; retracting the system by a selected length such that the distal portion of the first rigidizing device is exposed; and articulating the distal portion of the first rigidizing device to perform a loop (e.g., perform a loop motion with the tip) resulting in visualization by the camera of a circumference of a second portion of the body lumen, wherein at least a portion of the second portion is positioned proximally to the first portion. As used herein a circular pass movement may be a looping movement and is not limited to a circular path, but may be oval or irregular, though it may radially circumscribe the surrounding lumen. The circular pass movement may start and stop at approximately the same position; in some examples the circular pass movement extends beyond the radial starting position.

In any of these examples, the system may be retracted (or configured to retract) as described above, including retracting by a selected length such that the distal portion of the first rigidizing device is exposed.

In general, the apparatuses described herein may include a controller. A controller may include control circuitry, e.g., one or more processors (microprocessors), memory, timers, registers, etc.) and control logic, which may be software, hardware and/or firmware. These controllers may equivalently be referred to herein as “control circuitry.” Controllers may be implemented in software, firmware, hardware, or some suitable combination of at least two of the three.

In some examples the system may be configured to automatically perform the method. For example, the system may comprise a controller and a controller comprising one or more processors; and a memory coupled to the one or more processors, the memory storing computer-program instructions, that, when executed by the one or more processors, perform a computer-implemented method to advance the inner and outer rigidizing devices within a body lumen, and wherein the method is implemented by the system. The selected length may be calculated based on a desired distance from a portion of the lumen that has already been visualized by the camera. In some examples the first portion and the second portion may overlap.

Any of these methods may include compiling the data received by the camera to produce a model of at least a portion of the lumen. The method may include machine sensing to provide positioning information relating to a center of the lumen.

For example, described herein are systems including: a first rigidizing device positioned within a second rigidizing device; a controller comprising one or more processors; and a memory coupled to the one or more processors, the memory storing computer-program instructions, that, when executed by the one or more processors, perform a computer-implemented method to screen a body lumen, the method comprising: exposing a distal portion of the first rigidizing device; articulating the distal portion of the first rigidizing to perform a circular pass movement resulting in visualization by the camera of a circumference of a first portion of the body lumen; retracting the system by a selected length such that the distal portion of the first rigidizing device is exposed; and articulating the distal portion of the first rigidizing device to perform a circular pass movement (e.g., perform a rotating motion with the tip) resulting in visualization by the camera of a circumference of a second portion of the body lumen, at least a portion of the second portion positioned proximally to the first portion.

The selected length may be calculated based on a desired distance from a portion of the lumen has already been visualized by the camera. The first portion and the second portion may overlap.

The system may also include compiling the data received by the camera to produce a model of at least a portion of the lumen. Any of these systems may include machine sensing to provide positioning information on a center of the lumen.

All of the methods and apparatuses described herein, in any combination, are herein contemplated and can be used to achieve the benefits as described herein.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIG. 1 shows a rigidizing device.

FIGS. 2A-2B show exemplary rigidized shapes of a rigidizing device.

FIGS. 3A-3B show an example of a portion of a vacuum rigidizing apparatus as described herein. FIG. 3A shows a section through the exemplary vacuum rigidizing member of the apparatus. FIG. 3B shows an enlarged view of a portion of the section, illustrating the arrangement of layers in the un-rigidized configuration.

FIGS. 3C-3F show an example of a portion of a vacuum rigidizing apparatus having multiple rigidizing layers as described herein. FIG. 3C shows a perspective view of the vacuum rigidizing member with the outer layer removed (showing the outermost braid layer). FIG. 3D is an enlarged view of a portion of FIG. 3C. FIG. 3E shows a longitudinal section though the vacuum rigidizing member of FIG. 3C. FIG. 3F is a cross-section through the rigidizing member of FIG. 3C.

FIGS. 4A-4B show an exemplary pressure rigidizing device.

FIG. 5 shows a rigidizing device with a distal end section.

FIG. 6 shows a rigidizing device with a distal end section having a plurality of actively controlled linkages.

FIG. 7 shows a nested rigidizing system.

FIG. 8 shows a nested rigidizing system with a cover between the inner and outer rigidizing devices.

FIGS. 9A-9B show a nested rigidizing system where the outer rigidizing device includes steering and imaging.

FIGS. 10A-10H show exemplary use of a nested rigidizing system.

FIGS. 11A-11E schematically illustrate an example of using actuating steering members of a rigidizing device to aid in a transition of the rigidizing device from a rigidized to a flexible state.

FIGS. 12A-12E schematically illustrate an example of using actuating steering members of a rigidizing device to aid in a transition of the rigidizing device from a rigidized to a flexible state.

FIGS. 13A-13E schematically illustrate an example of using actuating steering members of a rigidizing device to aid in a transition of the rigidizing device from a rigidized to a flexible state.

FIGS. 14A-F show an exemplary method for performing a screening procedure in a body lumen using a nested rigidizing system.

FIGS. 15A-15C illustrates an example of a method for copying a shape between a nested pair of rigidizing devices.

FIGS. 16A-16D illustrates a method of controlling a nested pair of rigidizing devices to distally advance the nested by performing multiple shape copying sequences.

FIGS. 17A-17D illustrates a method of controlling a nested pair of rigidizing devices to proximally retract the nested pair including controlling the inner rigidizing device to assume a remembered shape in order to facilitate withdrawal of the inner rigidizing device.

FIGS. 18A-18F illustrate a method of controlling a nested pair of rigidizing devices including controlling of the tension on one or more steering actuators when transitioning between rigid and non-rigid states.

FIGS. 19A-19B illustrate a method of automatically shape copying between a nested pair of rigidizing devices when advancing one of the rigidizing device of the nested pair.

FIGS. 20A-20C illustrate a method of coordinating a roll of one rigidizing device of a nested pair of rigidizing devices.

FIGS. 21A-21B illustrate an example of a method of controlling the operation of a nested pair of rigidizing devices including automatically controlling movement of the nested pair.

FIGS. 22A-22B illustrate an example of a method of controlling the operation of a nested pair of rigidizing devices potentially leading to paradoxical movement.

FIGS. 23A-23B illustrate an example of a method of controlling the operation of a nested pair of rigidizing devices including automatic detection and correction for paradoxical movement.

### DETAILED DESCRIPTION

In general, described herein are nested rigidizing apparatuses (e.g., devices, system, etc.) that may be configured to navigate and/or aid in transporting a scope (e.g., endoscope) or other medical instrument through a curved or looped portion of the body, e.g., a portion of the gastrointestinal tract, including, but not limited to the colon, as well as methods of using them. In particular, described herein are methods and apparatuses for navigating the curvature of a body region, such as a colon, including both advancing and retracting, using a nested pair of rigidizing apparatuses.

The rigidizing devices described herein can be long, thin, and hollow (or solid) and can transition quickly from a flexible configuration (i.e., one that is relaxed, limp, or floppy) to a rigid configuration (i.e., one that is stiff and/or holds the shape it is in when it is rigidized). The rigidizing apparatus may include a plurality of layers (e.g., coiled or reinforced layers, slip layers, rigidizing layers, bladder layers and/or sealing sheaths) can together form the wall of the rigidizing devices, which may be referred to as “layered rigidizing apparatuses.” Unless the context makes clear otherwise, the methods and apparatuses described herein may refer to any appropriate rigidizing device, including layered rigidizing apparatuses. For example, the rigidizing devices (members, apparatuses, etc.) described herein may be rigidized by jamming particles, by phase change, by interlocking components (e.g., cables with discs or cones, etc.) or any other rigidizing mechanism. The rigidizing devices can transition from the flexible configuration to the rigid configuration, for example, by applying a vacuum or pressure to the wall of the rigidizing device or within the wall of the rigidizing device. With the vacuum or pressure removed, the layers can easily shear or move relative to each other. With the vacuum or pressure applied, the layers can transition to a condition in which they exhibit substantially enhanced ability to resist shear, movement, bending, torque and buckling, thereby providing system rigidization. Any of the apparatuses described herein may be configured for use in one or more of: the neurovasculature (e.g., aortic arch, subclavian, carotid, vertebral, basilar, posterior cerebral, circle of Willis, middle cerebral, anterior cerebral, etc.), the upper GI tract (mouth esophagus, stomach, pylorus, bile duct and pancreatic duct, etc.), the small bowel (e.g., small intestine, duodenum, jejunum, ilium, etc.), the lower GI tract (rectum, regions of colon, e.g., sigmoid, descending, transverse, ascending, cecum, ileocecal valve, etc.), the urinary tract (urethra, bladder, kidneys, ureters, etc.), the peripheral vasculature (e.g., femoral, iliac, mesenteric, lumbar, renal, celiac trunk, hepatic, thoracic, etc.), the cardiac region (e.g., aorta, right coronary artery, left coronary artery, etc.), the left heart (e.g., aorta, aortic valve, left ventricle, etc.), the right heart (e.g., vena cava, right atrium, left atrium, mitral valve, coronary sinus, tricuspid valve, right ventricle, pulmonary valve, pulmonary vasculature, etc.) and/or the right pulmonary region (e.g., mouth, larynx, trachea, bronchial tree and lobes etc.).

Any of the rigidizable apparatuses described herein may include rigidizing layers or regions that engage with a compression layer (which may be or may include a bladder) that applies force to the rigidizing layer to rigidize the rigidizing layer or in some cases to de-rigidize (e.g., release from rigidization) the rigidizing layer. In some examples, these rigidizable apparatuses may include a rigidizing layer that could include a braid, knit, woven, chopped segments, randomly distributed or randomly oriented filaments or strands, engagers, links, scales, plates, segments, particles, granules, crossing filaments, or other materials forming the rigidizing layer. For example, the rigidizing layer may comprise multiple strand lengths or strand segments that cross over each other (e.g., as part of a braid, knit, woven, etc.); the compression layer may apply force to drive the crossing strand lengths or strand segments against each other. Although many of the examples shown herein are braids, any of these apparatuses may instead or in addition include a general rigidizing layer comprising crossing strand lengths or strand segments.

The examples of rigidizing apparatuses described herein may use pressure (positive pressure) and/or negative pressure to selectively and controllable rigidize. In some examples the method described herein may be used with any appropriate rigidizing apparatus.

The rigidizing (e.g., selectively rigidizing) apparatuses described herein can provide rigidization for a variety of medical applications, including catheters, sheaths, scopes (e.g., endoscopes), wires, overtubes, trocars or laparoscopic instruments. The rigidizing devices can function as a separate add-on device or can be integrated into the body of catheters, sheaths, scopes, wires, or laparoscopic instruments. The devices described herein can also provide rigidization for non-medical structures.

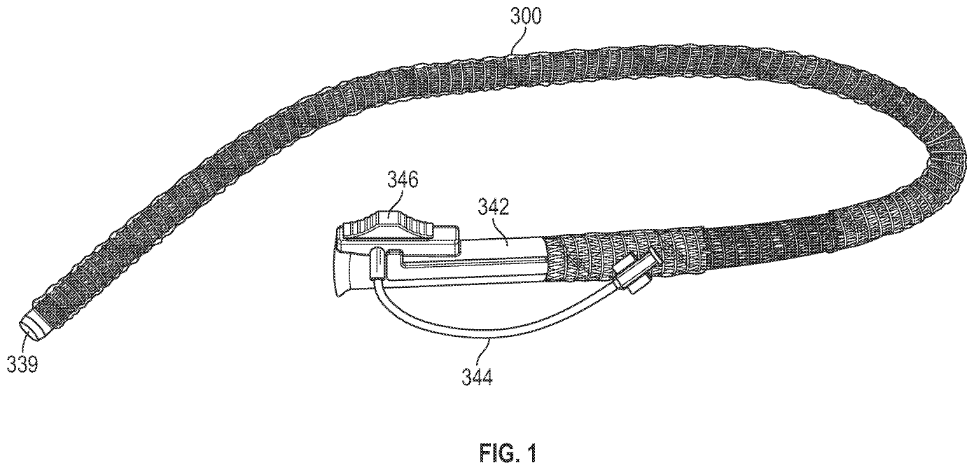

An exemplary rigidizing apparatus is shown in FIG. 1. The system shown includes a rigidizing device 300 having a wall with a plurality of layers including a rigidizing layer, an outer layer (part of which is cut away in this example to show the rigidizing layer thereunder, configured as a braid layer in this example), and an inner layer. The system further includes a handle 342 having a vacuum or pressure inlet 344 to supply vacuum or pressure to the rigidizing device 300. An actuation element 346 can be used to turn the vacuum or pressure on and off to thereby transition the rigidizing device 300 between flexible and rigid configurations. The distal tip 339 of the rigidizing device 300 can be smooth, flexible, and atraumatic to facilitate distal movement of the rigidizing device 300 through the body. Further, the tip 339 can taper from the distal end to the proximal end to further facilitate distal movement of the rigidizing device 300 through the body. In this example, the rigidizing apparatus is configured as an overtube, but other configurations may be used.

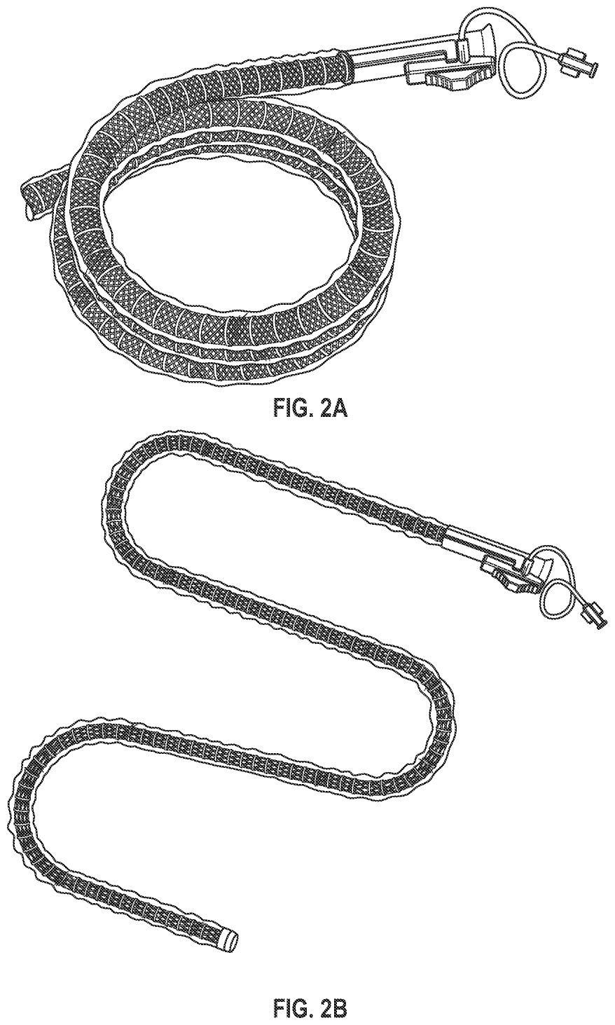

Exemplary rigidizing devices in a rigidized configuration are shown in FIGS. 2A and 2B. As the rigidizing device is rigidized, it locks into the shape it was in before vacuum or pressure was applied, i.e., it does not straighten, bend, or otherwise substantially modify its shape (e.g., it may stiffen in a looped configuration as shown in FIG. 2A or in a serpentine shape as shown in FIG. 2B). The air stiffening effect on the inner or outer layers (e.g., made of coil-wound tube) can be a small percentage (e.g., 5%) of the maximum load capability of the rigidizing device in bending, thereby allowing the rigidizing device to resist straightening. Upon release of the vacuum or pressure, strands within the rigidizing layer of the device can unlock relative to one another and again move so as to allow bending of the rigidizing device. Again, as the rigidizing device is made more flexible through the release of vacuum or pressure, it does so in the shape it was in before the vacuum or pressure was released, i.e., it does not straighten, bend, or otherwise substantially modify its shape. Thus, the rigidizing devices described herein can transition from a flexible, less-stiff configuration to a rigid configuration of higher stiffness by restricting the motion between the overlapping strands of rigidizing layers (e.g., braid layer), by applying vacuum or pressure.

The rigidizing apparatuses described herein can toggle between a rigid configuration and a flexible configuration quickly, and in some examples with an indefinite number of transition cycles. In some examples the degree of rigidization (e.g., the stiffness) of the apparatus may also be adjusted, for example, by adjusting the positive pressure (in examples that are rigidized by positive pressure) or vacuum (in examples rigidized by vacuum). As interventional medical devices are made longer and inserted deeper into the human body, and as they are expected to do more exacting therapeutic procedures, there is an increased need for precision and control. Selectively rigidizing devices (including selectively rigidizing overtubes) as described herein can advantageously provide both the benefits of flexibility (when needed) and the benefits of stiffness (when needed). Further, the rigidizing devices described herein can be used, for example, with classic endoscopes, colonoscopes, robotic systems, and/or navigation systems, such as those described in U.S. patent application Ser. No. 17/644,758, filed Dec. 16, 2021, titled “DEVICE FOR ENDOSCOPIC ADVANCEMENT THROUGH THE SMALL INTESTINE,” the entirety of which is incorporated by referenced herein.

The rigidizing devices described herein can additionally or alternatively include any of the features described with respect to U.S. patent application Ser. No. 17/644,758, filed Dec. 16, 2021, titled “DEVICE FOR ENDOSCOPIC ADVANCEMENT THROUGH THE SMALL INTESTINE,” U.S. patent application Ser. No. 16/631,473, filed on Jul. 19, 2018, titled “DYNAMICALLY RIGIDIZING OVERTUBE,” U.S. patent application Ser. No. 17/604,203, filed on Jan. 16, 2020, titled “DYNAMICALLY RIGIDIZING COMPOSITE MEDICAL STRUCTURES,” U.S. patent application Ser. No. 17/902,770, filed on Sep. 2, 2022, titled “NESTED RIGIDIZING DEVICES,” U.S. patent application Ser. No. 17/995,294, filed Mar. 29, 2021, titled “LAYERED WALLS FOR RIGIDIZING DEVICES,” and U.S. patent application Ser. No. 18/044,027, filed Sep. 3, 2021, titled “DYNAMICALLY RIGIDIZING GUIDERAIL AND METHODS OF USE,” the entireties of which are incorporated by reference herein.

The rigidizing devices described herein can be provided in multiple configurations, including different lengths and diameters. In some examples, the rigidizing devices can include working channels (for instance, for allowing the passage of typical endoscopic tools within the body of the rigidizing device), balloons, nested elements, and/or side-loading features.

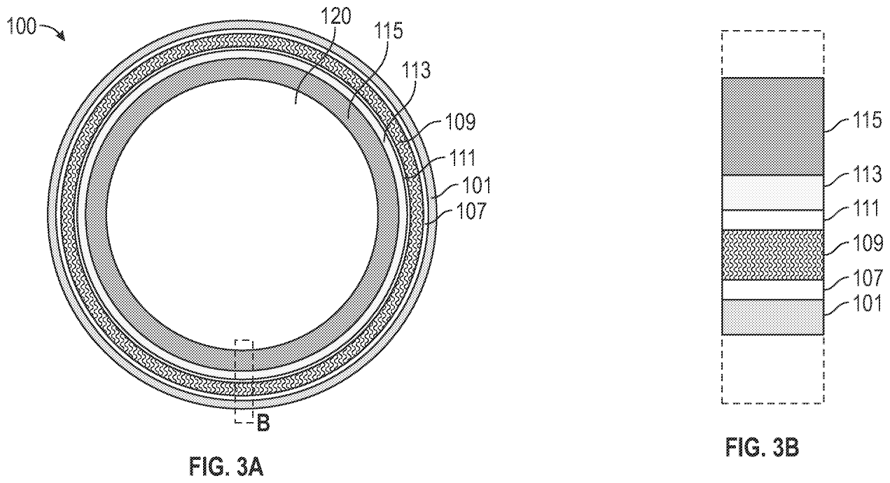

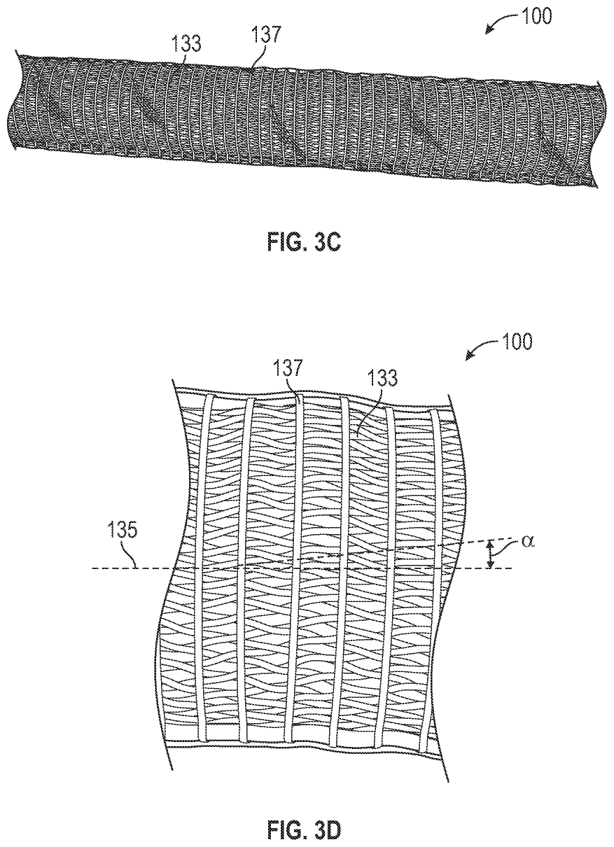

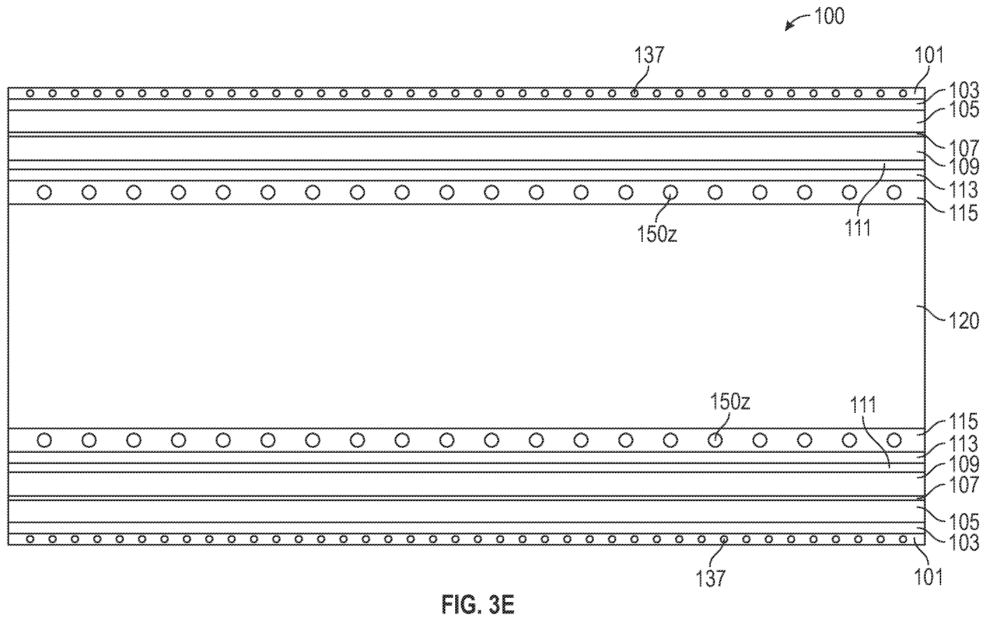

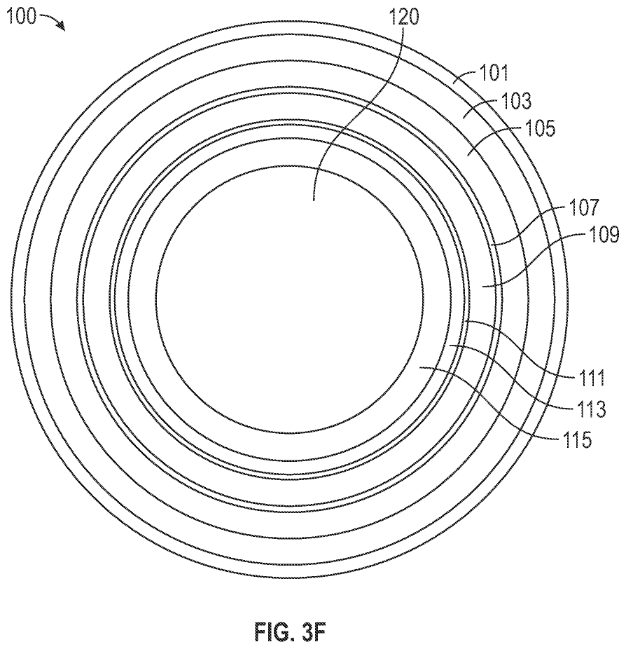

For example, a rigidizing apparatus 100 (also referred to as an apparatus, e.g., system and/or device, including a rigidizable member) may be configured to be rigidized by the application of vacuum, e.g., negative pressure. These apparatuses may generally be formed of layers that are configured to form a laminates structure when negative pressure is applied, so that one or more rigidizing layers may be reversibly fused to a flexible outer layer that is driven against a more rigid inner layer. FIGS. 3A-3B illustrate one example of a section through a rigidizing member of an apparatus (e.g., device, system) that is rigidized by the application of vacuum. FIG. 3B shows an enlarged view of the arrangement of the layers of FIG. 3A in the un-rigidized configuration. In this example, the rigidizable member includes an innermost layer 115 that is configured to provide an inner surface against which the remaining layers can be consolidated (e.g., when vacuum is applied). The innermost layer 115 can include a reinforcement element or coil. The rigidizing member may also include a slip layer 113 over (e.g., radially outwards of) the innermost layer. The slip layer may be, e.g., a lubrication, coating and/or powder (e.g., talcum powder) on the outer surface of the inner layer 115 and/or within the gap layer 111. A radial gap layer 111 may separate the slip layer 113 from a rigidizing layer (shown in this example as a braid or woven layer) 109 (referred to herein for convenience as a “rigidizing layer”), providing a space between the rigidizing layer and the slip layer for the rigidizing layer(s) thereover to move within, e.g., when no vacuum is applied; this space or gap may be removed when vacuum is applied, allowing the rigidizing layer(s) (e.g., in some examples a braided or woven layer) to move radially inward upon application of vacuum. A second gap layer 107 may be present between the rigidizing layer 109 and may be similar to layer 111. As will be described in reference to FIGS. 3C-3F, multiple rigidizing layers may be included (e.g., 2, 3 4 or more rigidizing layers may be included) and may be separated by additional gap layers and/or slip layers. The outermost layer 101 can be separated from the rigidizing layer(s) by a gap layer and can be configured to move radially inward when a vacuum is applied to pull down against the rigidizing layer(s) and conform onto the surface(s) thereof. The outermost layer 101 can be soft and atraumatic and can be sealed at both ends to create a vacuum-tight chamber with the innermost layer 115. The outermost layer 101 can be elastomeric, e.g., made of urethane. The hardness of the outermost layer 101 can be, for example, 30 A to 80 A. Further, the outermost layer 101 can have a thickness of 0.0001-0.01″, such as approximately 0.001″, 0.002, 0.003″ or 0.004″. Alternatively, the outermost layer can be plastic, including, for example, LDPE, nylon, or PEEK.

FIGS. 3C-3F illustrate an example of a tubular rigidizing member of an apparatus 100 that includes multiple rigidizing layers. As in FIGS. 3A-3B, the apparatus includes a tube having a wall formed of a plurality of layers positioned around a lumen 120 (e.g., for placement of an instrument or endoscope therethrough). A vacuum can be supplied between the layers to rigidize the rigidizing device 100. Any of the tubular apparatuses described herein may instead include a solid core forming the inner layer 115.

The innermost layer 115 can be configured to provide an inner surface against which the remaining layers can be consolidated, for example, when a vacuum is applied within the walls of the rigidizing device 100. The structure can be configured to minimize bend force and/or maximize flexibility in the non-vacuum condition. In some examples, the innermost layer 115 can include a reinforcement element 150z or coil within a matrix, as described above. In the example shown in FIG. 3E, the layer 113 over (i.e., radially outwards of) the innermost layer 115 can be a slip layer. The layer 111 can be a radial gap (i.e., a space). The gap layer 111 can provide space for the rigidizing layer(s) thereover to move within (when no vacuum is applied) as well as space within which the rigidizing layer(s) can move radially inward (upon application of vacuum).

The layer 109 can be a first rigidizing layer including, in this example, braided strands 133 similar to as described elsewhere herein. The rigidizing layer can be, for example, 0.001″ to 0.040″ thick. For example, a rigidizing layer can be 0.001″, 0.003″, 0.005″, 0.010″, 0.015″, 0.020″, 0.025″ or 0.030″ thick. In some examples, as shown in FIG. 3D, the rigidizing layer may comprise a braid having a tensile or hoop fibers 137. Hoop fibers 137 can be spiraled and/or woven into the rigidizing layer. Further, the hoop fibers 137 can be positioned at 2-50, e.g., 20-40 hoops per inch. The hoop fibers 137 can advantageously deliver high compression stiffness (to resist buckling or bowing out) in the radial direction but can remain compliant in the direction of the longitudinal axis 135 of the rigidizing device 100. That is, if compression is applied to the rigidizing device 100, the rigidizing layer 109 will try to expand in diameter as it compresses. The hoop fibers 137 can resist this diametrical expansion and thus resist compression. Accordingly, the hoop fiber 137 can provide a system that is flexible in bending but still resists both tension and compression.

The layer 107 can be another radial gap layer similar to layer 111.

In some examples, the rigidizing devices described herein can have more than one rigidizing layer. For example, the rigidizing devices can include two, three, or four rigidizing layers. Referring to FIG. 3E, the layer 105 can be a second rigidizing layer 105. The second rigidizing layer 105 can have any of the characteristics described with respect to the first rigidizing layer 109. In some examples, the second rigidizing layer 105 can be identical to the first rigidizing layer 109. In other examples, the second rigidizing layer 105 can be different than the of the first rigidizing layer 109. For example, in some examples the rigidizing layer is a braided layer; in FIG. 3E, the braid of the second braid layer 105 can include fewer strands and have a larger braid angle α than the braid of the first braid layer 109. Having fewer strands can help increase the flexibility of the rigidizing device 100 (relative to having a second strand with equivalent or greater number of strands), and a larger braid angle α can help constrict the diameter of the of the first braid layer 109 (for instance, if the first braid layer is compressed) while increasing/maintaining the flexibility of the rigidizing device 100. As another example, the braid of the second braid layer 105 can include more strands and have a larger braid angle α than the braid of the first braid layer 109. Having more strands can result in a relatively tough and smooth layer while having a larger braid angle α can help constrict the diameter of the first braid layer 109.

The layer 103 can be another radial gap layer similar to layer 111. The gap layer 103 can have a thickness of 0.0002-0.04″, such as approximately 0.03″. A thickness within this range can ensure that the strands 133 of the rigidizing layer(s) can easily slip and/or bulge relative to one another to ensure flexibility during bending of the rigidizing device 100.

The outermost layer 101 can be configured to move radially inward when a vacuum is applied to pull down against the rigidizing layers 105, 109 and conform onto the surface(s) thereof. The outermost layer 101 can be soft and atraumatic and can be sealed at both ends to create a vacuum-tight chamber with layer 115. The outermost layer 101 can be elastomeric, e.g., made of urethane. The hardness of the outermost layer 101 can be, for example, 30 A to 80 A. Further, the outermost layer 101 can have a thickness of 0.0001-0.01″, such as approximately 0.001″, 0.002, 0.003″ or 0.004″. Alternatively, the outermost layer can be plastic, including, for example, LDPE, nylon, or PEEK.

In some examples, the outermost layer 101 can, for example, have tensile or hoop fibers 137 extending therethrough. The hoop fibers 137 can be made, for example, of aramids (e.g., Technora, nylon, Kevlar), Vectran, Dyneema, carbon fiber, fiber glass or plastic. Further, the hoop fibers 137 can be positioned at 2-50, e.g., 20-40 hoops per inch. In some examples, the hoop fibers 137 can be laminated within an elastomeric sheath. The hoop fibers can advantageously deliver higher stiffness in one direction compared to another (e.g., can be very stiff in the hoop direction, but very compliant in the direction of the longitudinal axis of the rigidizing device). Additionally, the hoop fibers can advantageously provide low hoop stiffness until the fibers are placed under a tensile load, at which point the hoop fibers can suddenly exhibit high hoop stiffness.

In some examples, the outermost layer 101 can include a lubrication, coating and/or powder (e.g., talcum powder) on the outer surface thereof to improve sliding of the rigidizing device through the anatomy. The coating can be hydrophilic (e.g., a Hydromer® coating or a Surmodics® coating) or hydrophobic (e.g., a fluoropolymer). The coating can be applied, for example, by dipping, painting, or spraying the coating thereon.

The innermost layer 115 can similarly include a lubrication, coating (e.g., hydrophilic or hydrophobic coating), and/or powder (e.g., talcum powder) on the inner surface thereof configured to allow the bordering layers to more easily shear relative to each other, particularly when no vacuum is applied to the rigidizing device 100, to maximize flexibility.

In some examples, the outermost layer 101 can be loose over the radially inward layers. For instance, the inside diameter of layer 101 (assuming it constitutes a tube) may have a diametrical gap of 0″-0.200″ with the next layer radially inwards (e.g., with a rigidizing layer). This may give the vacuum rigidized system more flexibility when not under vacuum while still preserving a high rigidization multiple. In other examples, the outermost layer 101 may be stretched some over the next layer radially inwards (e.g., the rigidizing layer). For instance, the zero-strain diameter of a tube constituting layer 101 may be from 0-0.200″ smaller in diameter than the next layer radially inwards and then stretched thereover. When not under vacuum, this system may have less flexibility than one wherein the outer layer 101 is looser. However, it may also have a smoother outer appearance and be less likely to tear during use.

In some examples, the outermost layer 101 can be loose over the radially inward layers. A small positive pressure may be applied underneath the layer 101 in order to gently expand layer 101 and allow the rigidizing device to bend more freely in the flexible configuration. In this example, the outermost layer 101 can be elastomeric and can maintain a compressive force over the rigidizing layer, thereby imparting stiffness. Once positive pressure is supplied (enough to nominally expand the sheath off of the rigidizing layer, for example, 2 psi), the outermost layer 101 is no longer a contributor to stiffness, which can enhance baseline flexibility. Once rigidization is desired, positive pressure can be replaced by negative pressure (vacuum) to deliver stiffness.

A vacuum can be carried within rigidizing device 100 from minimal to full atmospheric vacuum (e.g., approximately 14.7 psi). In some examples, there can be a bleed valve, regulator, or pump control such that vacuum is bled down to any intermediate level to provide a variable stiffness capability. The vacuum pressure can advantageously be used to rigidize the rigidizing device structure by compressing the layer(s) of rigidizing layer (e.g., a braided sleeve) against neighboring layers. The rigidizing layer, such as a braid, knit or woven material, may be naturally flexible in bending (i.e. when bent normal to its longitudinal axis), and the lattice structure formed by the interlaced strands distort as the sleeve is bent in order for the rigidizing layer to conform to the bent shape while resting on the inner layers. In some examples, this results in lattice geometries where the corner angles of each lattice element change as the braided sleeve bends. When compressed between conformal materials, such as the layers described herein, the lattice elements become locked at their current angles and have enhanced capability to resist deformation upon application of vacuum, thereby rigidizing the entire structure in bending when vacuum is applied. Further, in some examples, the hoop fibers through or over the braid can carry tensile loads that help to prevent local buckling of the braid at high applied bending load.

The stiffness of the rigidizing device 100 can increase from 2-fold to over 30-fold, for instance 10-fold, 15-fold, or 20-fold, when transitioned from the flexible configuration to the rigid configuration. In one specific example, the stiffness of a rigidizing device similar to rigidizing device 100 was tested. The wall thickness of the test rigidizing device was 1.0 mm, the outer diameter was 17 mm, and a force was applied at the end of a 9.5 cm long cantilevered portion of the rigidizing device until the rigidizing device deflected 10 degrees. The forced required to do so when in flexible mode was only 30 grams while the forced required to do so in rigid (vacuum) mode was 350 grams.

In some examples of a vacuum rigidizing device 100, there can be only one rigidizing layer. In other examples of a vacuum rigidizing device 100, there can be two, three, or more rigidizing layers. In some examples, one or more of the radial gap layers or slip layers of rigidizing device 100 can be removed. In some examples, some or all of the slip layers of the rigidizing device 100 can be removed.

The rigidizing layers described herein can act as a variable stiffness layer. The variable stiffness layer can include one or more variable stiffness elements or structures that, when activated (e.g., when vacuum is applied), the bending stiffness and/or shear resistance is increased, resulting in higher rigidity. Other variable stiffness elements can be used in addition to or in place of the rigidizing layer. In some examples, engagers can be used as a variable stiffness element, as described in International Patent Application No. PCT/US2018/042946, filed Jul. 19, 2018, titled “DYNAMICALLY RIGIDIZING OVERTUBE,” the entirety of which is incorporated by reference herein. Alternatively or additionally, the variable stiffness element can include particles or granules, jamming layers, scales, rigidizing axial members, rigidizers, longitudinal members or substantially longitudinal members.

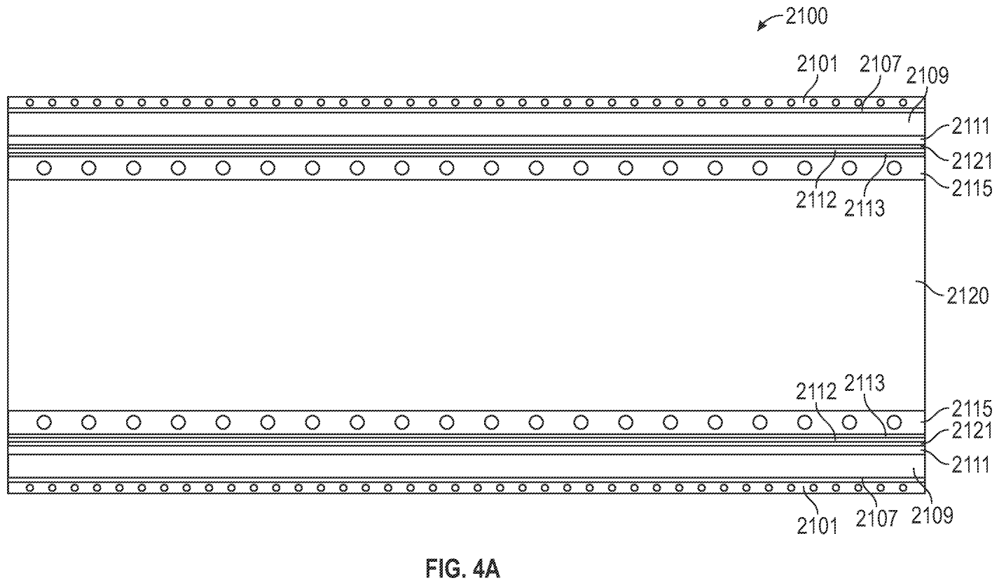

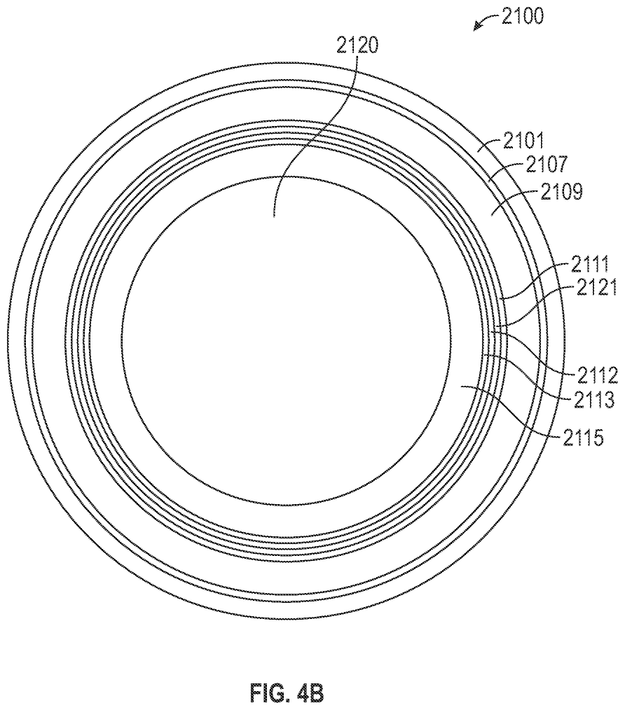

The rigidizable apparatuses described herein may also be rigidized by the application of positive pressure, rather than vacuum. For example, referring to FIGS. 4A-4B, the rigidizing apparatus (e.g., device or system) 2100 can be similar to rigidizing apparatus 100 described above, except that it can be configured to hold pressure (e.g., of greater than 1 atm) therein for rigidization rather than vacuum. A pressure-activated rigidizing device 2100 can also include a plurality of layers positioned around a lumen 2120 (e.g., for placement of an instrument or endoscope therethrough).

For example, FIGS. 4A-4B illustrate longitudinal and radial sections through an example of a pressure-activated rigidizable member of a rigidizing apparatus. The rigidizing device 2100 shown in FIGS. 4A and 4B can include an innermost layer 2115 (similar to innermost layer 115), a slip layer 2113 (similar to slip layer 113), a pressure gap 2112, a bladder layer 2121, a gap layer 2111 (similar to gap layer 111), a rigidizing layer 2109 (similar to rigidizing layer 109, e.g., a braid layer) or other variable stiffness layer as described herein, a gap layer 2107 (similar to layer 107), and an outermost containment layer 2101.

The pressure gap 2112 can be a sealed chamber that provides a gap for the application of pressure to layers of rigidizing device 2100. The pressure can be supplied to the pressure gap 2112 using a fluid or gas inflation/pressure media. The inflation/pressure media can be water or saline or, for example, a lubricating fluid such as oil or glycerin. The lubricating fluid can, for example, help the layers of the rigidizing device 2100 flow over one another in the flexible configuration. The inflation/pressure media can be supplied to the gap 2112 during rigidization of the rigidizing device 2100 and can be partially or fully evacuated therefrom to transform the rigidizing device 2100 back to the flexible configuration. In some examples, the pressure gap 2112 of the rigidizing device 2100 can be connected to a pre-filled pressure source, such as a pre-filled syringe or a pre-filled insufflator, thereby reducing the physician's required set-up time.

The bladder layer 2121 can be made, for example, of a low durometer elastomer (e.g., of shore 20 A to 70 A) or a thin plastic sheet. The bladder layer 2121 can be formed out of a thin sheet of plastic or rubber that has been sealed lengthwise to form a tube. The lengthwise seal can be, for instance, a butt or lap joint. For instance, a lap joint can be formed in a lengthwise fashion in a sheet of rubber by melting the rubber at the lap joint or by using an adhesive. In some examples, the bladder layer 2121 can be 0.0002-0.020″ thick, such as approximately 0.005″ thick. The bladder layer 2121 can be soft, high-friction, stretchy, and/or able to wrinkle easily. In some examples, the bladder layer 2121 is a polyolefin or a PET. The bladder 2121 can be formed, for example, by using methods used to form heat shrink tubing, such as extrusion of a base material and then wall thinning with heat, pressure and/or radiation. When pressure is supplied through the pressure gap 2112, the bladder layer 2121 can expand through the gap layer 2111 to push the rigidizing layer 2109 against the outermost containment layer 2101 such that the relative motion of the rigidizing layer strands is reduced.

The outermost containment layer 2101 can be a tube, such as an extruded tube. Alternatively, the outermost containment layer 2101 can be a tube in which a reinforcing member (for example, metal wire, including round or rectangular cross-sections) is encapsulated within an elastomeric matrix, similar to as described with respect to the innermost layer for other examples described herein. In some examples, the outermost containment layer 2101 can include a helical spring (e.g., made of circular or flat wire), and/or a tubular rigidizing layer (such as one made from round or flat metal wire) and a thin elastomeric sheet that is not bonded to the other elements in the layer. The outermost containment layer 2101 can be a tubular structure with a continuous and smooth surface. This can facilitate an outer member that slides against it in close proximity and with locally high contact loads (e.g., a nested configuration as described further herein). Further, the outer layer 2101 can be configured to support compressive loads, such as pinching. Additionally, the outer layer 2101 (e.g., with a reinforcement element therein) can be configured to prevent the rigidizing device 2100 from changing diameter even when pressure is applied.

Because both the outer layer 2101 and the inner layer 2115 include reinforcement elements therein, the rigidizing layer 2109 can be reasonably constrained from both shrinking diameter (under tensile loads) and growing in diameter (under compression loads).

By using pressure rather than vacuum to transition from the flexible state to the rigid state, the rigidity of the rigidizing device 2100 can be increased. For example, in some examples, the pressure supplied to the pressure gap 2112 can be between 1 and 40 atmospheres, such as between 2 and 40 atmospheres, such as between 4 and 20 atmospheres, such as between 5 and 10 atmospheres. In some examples, the pressure supplied is approximately 2 atm, approximately 4 atmospheres, approximately 5 atmospheres, approximately 10 atmospheres, approximately 20 atmospheres. In some examples, the rigidizing device 2100 can exhibit change in relative bending stiffness (as measured in a simple cantilevered configuration) from the flexible configuration to the rigid configuration of 2-100 times, such as 10-80 times, such as 20-50 times. For example, the rigidizing device 2100 can have a change in relative bending stiffness from the flexible configuration to the rigid configuration of approximately 10, 15, 20, or 25, 30, 40, 50, or over 100 times.

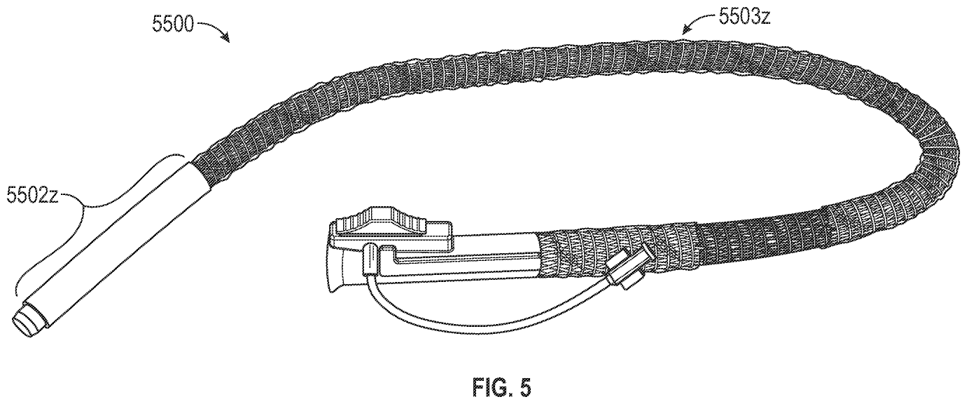

Any of the rigidizing devices described herein can have a distal end section or sections with a different design than the main elongate body of the rigidizing device. As shown in FIG. 5, for example, rigidizing device 5500 can have a main elongate body 5503z and a distal end section 5502z. Only the distal end section 5502z, only the main elongate body 5503z, or both the distal end section 5502z and the main elongate body 5503z can be rigidizing as described herein (e.g., by vacuum and/or pressure). In some examples, one section 5502z, 5503z is activated by pressure and the other section 5502z, 5503z is activated by vacuum. In other examples, both sections 5502z, 5503z are activated by pressure or vacuum, respectively.

Any of the rigidizing devices (e.g., the inner and/or outer rigidizing devices) may be configured to be steered (e.g., controllably bent or curved), particularly at their distal end regions. Any of these apparatuses may include one or more actuating steering members that are configured to be actually, e.g., from a proximal end of the device, to steer the device. The actuating steering members may be any appropriate steering member, including mechanical steering (e.g., one or more tendons, cables, wires, etc., actuators, etc.), pneumatic steering, magnetic steering, thermal steering (e.g., using a shape memory alloy or shape memory polymers, etc.). Although the examples described herein include primarily actuating steering members comprising one or more cables, any appropriate actuating steering member may be used in any of these apparatuses and methods.

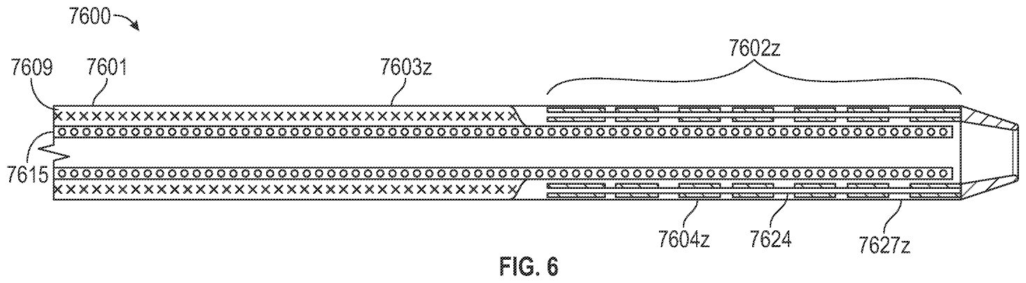

Referring to FIG. 6, in other examples, the distal end section 7602z can include a plurality of linkages 7604z that are actively controlled, such as via actuating steering members (e.g., cables 7624), for steering of the rigidizing device 7600. The device 7600 is similar to device 5800 except that it includes cables 7624 configured to control movement of the device. While the passage of the cables 7624 through the rigidizing elongate body 7603z (i.e., with outer wall 7601, rigidizing layer 7609, and inner layer 7615) is not shown in FIG. 26, the cables 7624 can extend therethrough in any manner as described elsewhere herein. In some examples, one or more layers of the rigidizing elongate body 7603z can continue into the distal end section 7602z. For example, and as shown in FIG. 26, the inner layer 7615 can continue into the distal end section 7602z, e.g., can be located radially inwards of the linkages 7604z. Similarly, any of the additional layers from the rigidizing proximal section (e.g., the rigidizing layer 7609 or the outer layer 7601 may be continued into the distal section 7602z and/or be positioned radially inwards of the linkages 7604z). In other examples, none of the layers of the rigidizing elongate body 7603z continue into the distal section 7602z. The linkages 7604z (and any linkages described herein) can include a covering 7627z thereover. The covering 7627z can advantageously make the distal section 7602z atraumatic and/or smooth. The covering 7627z can be a film, such as expanded PTFE. Expanded PTFE can advantageously provide a smooth, low friction surface with low resistance to bending but high resistance to buckling.

In some examples, the rigidizing devices described herein can be used in conjunction with one or more other rigidizing devices described herein. For example, an endoscope can include the rigidizing mechanisms described herein, and a rigidizing device can include the rigidizing mechanisms described herein. Used together, they can create a nested system that can advance, one after the other, allowing one of the elements to always remain stiffened, such that looping is reduced or eliminated (i.e., they can create a sequentially advancing nested system).

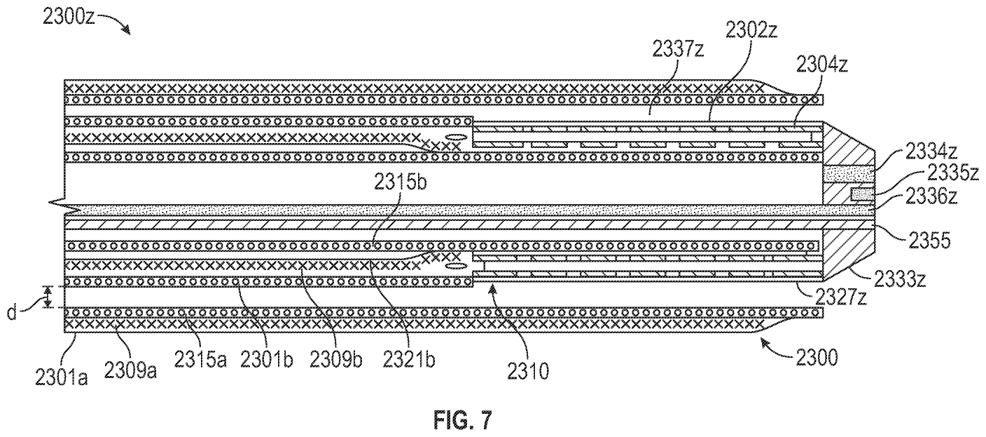

An exemplary nested system 2300z is shown in FIG. 7. The system 2300z can include an outer rigidizing device 2300 and an inner rigidizing device 2310 (here, configured as a rigidizing scope) that are axially movable with respect to one another either concentrically or non-concentrically. The outer rigidizing device 2300 and the inner rigidizing device 2310 can include any of the rigidizing features as described herein. For example, the outer rigidizing device 2300 can include an outermost layer 2301a, a rigidizing layer 2309a, and an inner layer 2315a including a coil wound therethrough. The outer rigidizing device 2300 can be, for example, configured to receive vacuum between the outermost layer 2301a and the inner layer 2315a to provide rigidization. Similarly, the inner scope 2310 can include an outer layer 2301b (e.g., with a coil wound therethrough), a rigidizing layer 2309b, a bladder layer 2321b, and an inner layer 2315b (e.g., with a coil wound therethrough). The inner scope 2310 can be, for example, configured to receive pressure between the bladder 2321b and the inner layer 2315b to provide rigidization. Further, an air/water channel 2336z and a working channel 2355 can extend through the inner rigidizing device 2310. Additionally, the inner rigidizing scope 2310 can include a distal section 2302z with a camera 2334z, lights 2335z, and steerable linkages 2304z. A cover 2327z can extend over the distal section 2302z. In another example, the camera and/or lighting can be delivered in a separate assembly (e.g., the camera and lighting can be bundled together in a catheter and delivered down the working channel 2355 and/or an additional working channel to the distalmost end 2333z).

An interface 2337z can be positioned between the inner rigidizing device 2310 and the outer rigidizing device 2300. The interface 2337z can be a gap, for example, having a dimension d (see FIG. 5) of 0.001″-0.050″, such as 0.0020″, 0.005″, or 0.020″ thick. In some examples, the interface 2337z can be low friction and include, for example, powder, coatings, or laminations to reduce the friction. In some examples, there can be seals between the inner rigidizing device 2310 and outer rigidizing device 2300, and the intervening space can be pressurized, for example, with fluid or water, to create a hydrostatic bearing. In other examples, there can be seals between the inner rigidizing device 2310 and outer rigidizing device 2300, and the intervening space can be filled with small spheres to reduce friction.

The inner rigidizing device 2310 and outer rigidizing device 2300 can move relative to one another and alternately rigidize so as to transfer a bend or shape down the length of the nested system 2300z. For example, the inner device 2310 can be inserted into a lumen and bent or steered into the desired shape. Pressure can be applied to the inner rigidizing device 2310 to cause the rigidizing layer elements to engage and lock the inner rigidizing device 2310 in the configuration. The rigidizing device (for instance, in a flexible state) 2300 can then be advanced over the rigid inner device 2310. When the outer rigidizing device 2300 reaches the tip of the inner device 2310, vacuum can be applied to the rigidizing device 2300 to cause the layers to engage and lock to fix the shape of the rigidizing device. The inner device 2310 can be transitioned to a flexible state, advanced, and the process repeated. Although the system 2300z is described as including a rigidizing device and an inner device configured as a scope, it should be understood that other configurations are possible. For example, the system might include two overtubes, two catheters, or a combination of overtube, catheter, and scope.

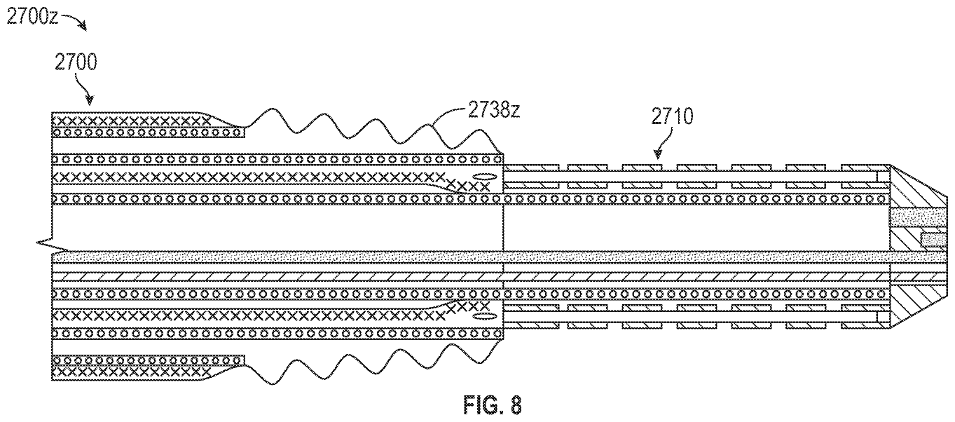

FIG. 8 shows another exemplary nested system 2700z. System 2700z is similar to system 2300z except that it includes a cover 2738z attached to both the inner and outer rigidizing device 2710, 2700. The cover 2738z may be, for example, low-durometer and thin-walled to allow elasticity and stretching. The cover 2738z may be a rubber, such as urethane, latex, or silicone. The cover 2738z may protect the interface/radial gap between the inner and outer devices 2710, 2700. The cover 2738z may prevent contamination from entering the space between the inner and outer tubes. The cover 2738z may further prevent tissue and other substances from becoming trapped in the space between the inner and outer tubes. The cover 2738z may stretch to allow the inner device 2710 and outer device 2700 to travel independently of one another within the elastic limits of the material. The cover 2738z may be bonded or attached to the rigidizing devices 2710, 2700 in such a way that the cover 2738z is always at a minimum slightly stretched. This example may be wiped down externally for cleaning. In some examples, the cover 2738z can be configured as a “rolling” seal, such as disclosed in U.S. Pat. No. 6,447,491, the entire disclosure of which is incorporated by reference herein.

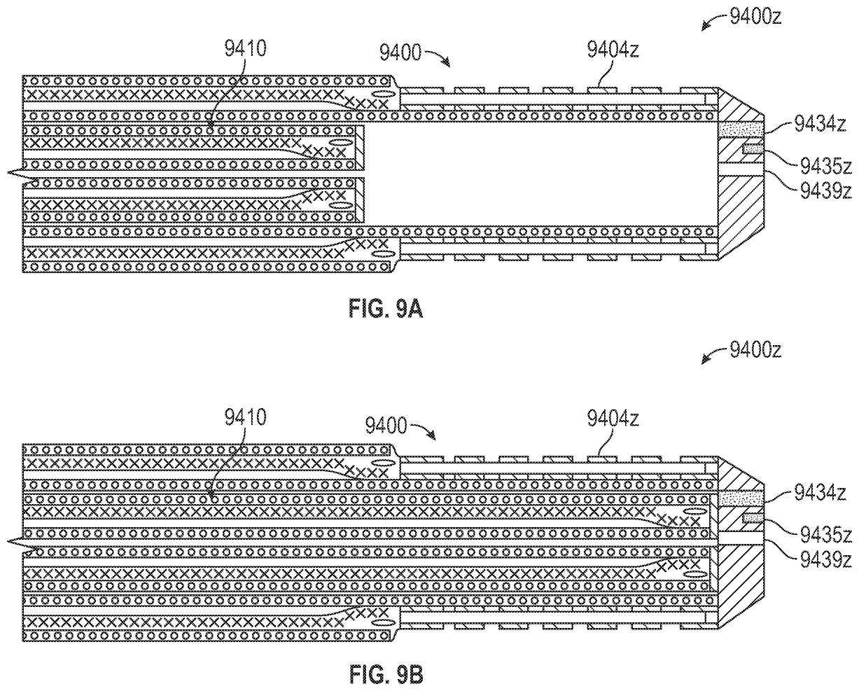

FIGS. 9A-9B show another exemplary nested system 9400z. In this system 9400z, the outer rigidizing device 9400 includes steering and imaging (e.g., similar to a scope) while the inner device includes only rigidization (though it could include additional steering elements as described elsewhere herein). Thus, outer device 9400 includes linkages or other steering means disclosed herein 9404z, camera 9434z, and lighting 9435z. The outer device 9400 can further include a central passageway 9439z for access to the inner device 9410 (e.g., lumens such as working channels therein). In some examples, bellows or a loop of tubing can connect the passageway 9439z to lumens of the inner device 9410. Similar to the other nested systems, at least one of the devices 9410, 9400 can be rigidized at a time while the other can conform to the rigidization and/or move through the anatomy. Here, the outer device 9400 can lead the inner device 9410 (the inner device 9410 is shown retracted relative to the outer device 9400 in FIG. 9A and extended substantially even with the outer device 9400 in FIG. 7B). Advantageously, system 9400z can provide a smooth exterior surface to avoid pinching the anatomy and/or entrance of fluid between the inner and outer devices 9410, 9400. Having the steering on the outer device 9400 can also provide additional leverage for steering the tip. Also, the outer device can facilitate better imaging capabilities due to the larger diameter of the outer device 9400 and its ability to accommodate a larger camera.

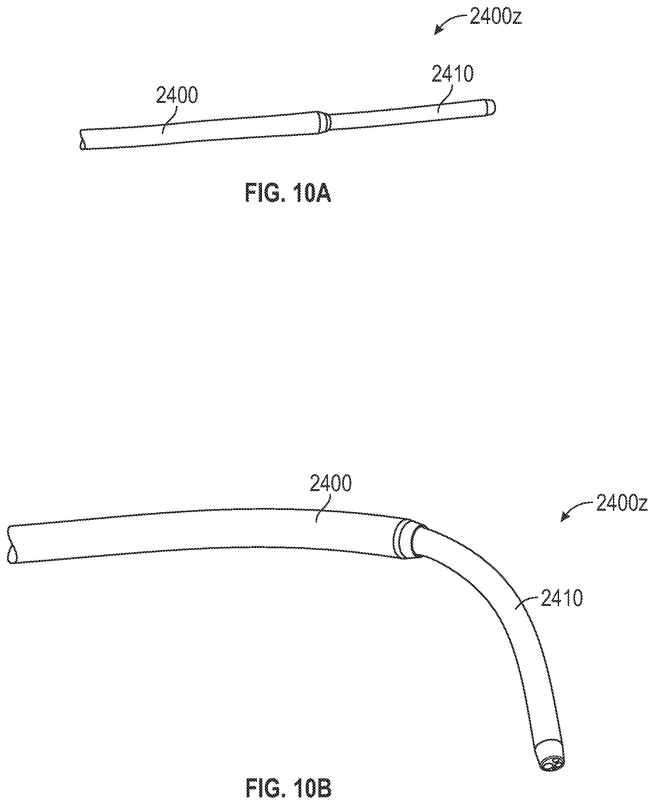

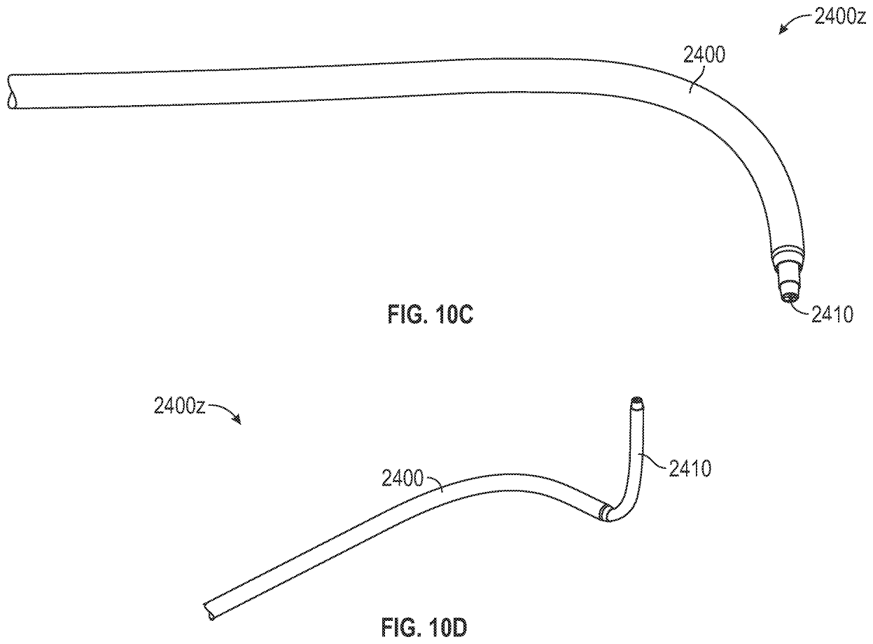

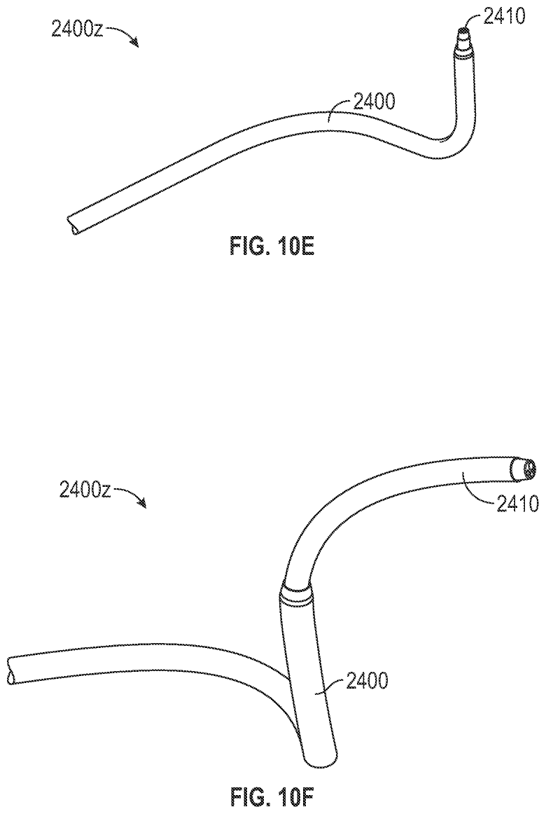

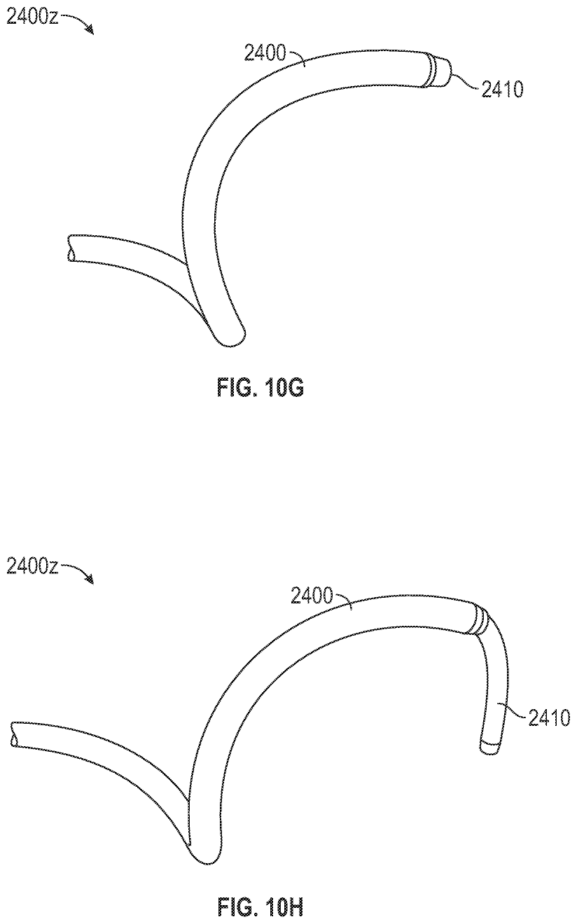

FIGS. 10A-10H show the exemplary use of a nested system 2400z as described herein. At FIG. 10A, a steerable inner rigidizing device 2410 is positioned within the outer rigidizing device 2400 such that the distal end of the inner rigidizing device 2410 extends outside of the outer rigidizing device 2400. At FIG. 10B, the distal end of the inner rigidizing device 2410 is bent in the desired direction/orientation (e.g., via actuating steering members, such as cables 7624) and then rigidized (e.g., using vacuum or pressure as described herein). At FIG. 10C, the outer rigidizing device 2400 (in the flexible configuration) is advanced over the rigidized inner rigidizing device 2410 (including over the bending distal section). Once the distal end of the outer rigidizing device 2400 is sufficiently advanced over the distal end of the inner rigidizing device 2410, then the outer rigidizing device 2400 can be rigidized (e.g., using vacuum or pressure as described herein). At FIG. 10D, the inner rigidizing device 2410 can then be transitioned to the flexible state (e.g., by removing the vacuum or pressure as described herein and by allowing the steering cables to go slack such that tip can move easily) and can be advanced and directed/oriented/steered as desired. Alternately, in FIG. 10D, the inner rigidizing device 2410 can be actively steered (either manually or via computational control) as it emerges such that it minimizes the load on the rigidized outer tube. Minimizing the load on the outer rigidizing device 2400 makes it easier for this tube to hold the rigidized shape. Once the inner rigidizing device 2410 is rigidized, the outer rigidizing device 2400 can be transitioned to the flexible state and advanced thereover (as shown in FIG. 10E). The process can then be repeated as shown in FIGS. 10F-H. The repeated process can result in “shape copying,” whereby the inner and outer rigidizing devices 2410, 2400 in the flexible configuration continuously conform to (or copy) the shape of whichever device 2410, 2400 is in the rigid configuration. In some examples, at the completion of the sequence shown in FIGS. 10A-H, a third rigidizing device can be slid over the first two rigidizing devices (2400, 2410) and rigidized. Rigidizing devices 2400 and 2410 can then be withdrawn. Finally, a fourth rigidizing device can be inserted through the inner lumen of the third tube. This fourth rigidizing device may have a larger diameter and more features than rigidizing device 2410. For instance, it may have a larger working channel, more working channels, a better camera, or combinations thereof. This technique can allow two smaller tubes, which tend to be more flexible and maneuverable, to reach deep into the body while still ultimately deliver a larger tube for therapeutic purposes. Alternatively, in the example above, the fourth rigidizing device can be a regular endoscope as is known in the art.

In some examples, at the completion of the sequence shown in FIGS. 10A-H, outer rigidizing device 2400 may be rigidized and then the inner rigidizing device 2410 may be removed. For example, the rigidizing device 2410 may be a “navigation” device comprising a camera, lighting and a distal steering section. The “navigation” device 2410 may be well sealed such that it is easy to clean between procedures. A second inner device may then be placed inside the rigidized outer device 2400 and advanced past the distal end of the outer device 2400. The second inner device may be a “therapeutic” tube comprising such elements as a camera, lights, water, suction and various tools. The “therapeutic” device may not have a steering section or the ability to rigidize, thereby giving additional room in the body of the therapeutic tube for the inclusion of other features, for example, tools for performing therapies. Once in place, the tools on the “therapeutic” tube may be used to perform a therapy in the body, such as, for example, a mucosal resection or dissection in the human GI tract.

In another example, after or during the completion of the sequence shown in FIGS. 10A-H, a third device may be inserted inside inner tube 2410. The third device may be rigidizing and/or an endoscope.

In some examples, after completion of the sequence shown in FIGS. 10A-10H and the completion of any therapies conducted with the system 2400z in place, the entire system 2400z can be removed from the anatomy. In one exemplary method of withdrawing, the system 2400z can be transitioned to the flexible configuration (i.e., both the inner and outer devices 2410, 2400 can be transitioned to the flexible configuration), and the flexible system 2400z can be pulled proximally. In this method, the tension between the patient's body (e.g., the anus) and a robotic arm (e.g., arm 1023y described below) can prevent the system 2400z from falling out of the body as it is removed (e.g., as more of the flexible system 2400z is positioned outside of the body than inside of the body).

As another exemplary method of withdrawing, shape copying can be performed similar to as described with respect to FIGS. 10A-10H, but in reverse. In this example, for example, the inner rigidizing device 2410 can be rigidized and the outer rigidizing device 2400 can be withdrawn proximally (while in the flexible configuration) over the inner rigidizing device 2410. The outer rigidizing device 2400 can then be rigidized and the inner rigidizing device 2410 can be relaxed and moved proximally within the outer rigidizing device 2400 (e.g., until the distal end of the inner rigidizing device 2410 is flush with the distal end of the outer rigidizing device 2400). In this example, when the inner rigidizing device 2410 is withdrawn into the outer rigidizing device 2400, tension on the steering cables can be held constant (e.g., at a low value, such as ¼ lb or less) to ensure that the steerable distal end section will move into the shape of the outer rigidizing device 2400 without disturbing the fixed shape of the outer rigidizing device 2400. Alternatively or additionally, if the outer rigidizing device 2400 is rigidized in a straight shape, then the inner rigidizing device 2410 can be pulled into the outer rigidizing device 2400 and tension on each of the steering cables can be made equal (i.e., the same value, thus conforming the child shape to shape of the inside of the mother).

As another exemplary method of withdrawing, the steerable distal tip of the inner rigidizing device 2410 can be actively steered proximally into the known, assumed, or measured shape of the outer rigidizing device 2400 either as or after the distal tip is retracted into the outer rigidizing device 2410. That is, the distal tip of the inner rigidizing device 2410 can be steered to match the shape of the section of the outer rigidizing device 2400 that is immediately proximal to the distal tip of the inner rigidizing device 2410. In one specific example, the inner rigidizing device 2410 may project from the outer rigidizing device 2400 by 4 inches, and the last 4 inches of the outer rigidizing device 2400 may form a 90 degree curve around a 2.5 inch radius of curvature. In this example, the inner rigidizing device 2410 can be steered into a 90 degree curve around a 2.5 inch radius of curvature and then withdrawn (in that shape) into the outer rigidizing device 2400. This may advantageously ensure that the inner rigidizing device 2410 pulls easily into the outer rigidizing device 2400 (i.e., because their shapes are matched).

In some examples, certain methods, controls, and/or algorithms can be used to enhance the advancement or withdrawal of nested rigidizing devices like those described herein. As described above, during advancing or withdrawing a nested system, the devices are alternately made flexible and rigidized to travel along the body lumen. Once the flexible device is advanced over or within the rigidized device and the flexible device copies the shape of the rigidized device, the rigidized device may then be made flexible to be advanced or withdrawn. In some examples, actuating steering members (e.g., such as steering cables 7624) can be used to control the shape of the rigidized device and/or to maintain control of the device as/when it moves to its flexible state.

For example, when advancing a nested system, after the outer rigidizing device (e.g. outer rigidizing device 2400) has been advanced over the inner rigidizing device and has copied its shape, the inner rigidizing device (e.g., inner rigidizing device 2410) is transitioned to a flexible state prior to its advancement. In some examples, at the point shown in FIG. 10D, the inner rigidizing device can be steered to maintain the previously commanded curvature of the inner rigidizing device. The previously commanded curvature can refer to the curvature imposed by the actuating steering member(s) prior to rigidization of the inner device.

Maintaining the previously commanded curvature can have advantages over allowing the inner rigidizing device to transition to the flexible state with the steering cables slack. For example, during a partial copy of the inner rigidizing device, in which the outer rigidizing device is not fully advanced over the inner rigidizing device, it may be undesirable to allow the exposed length of the inner device to straighten at the completion of the partial copy. In some examples, in the absence of either rigidization or tension from the cables, the inner rigidizing device may tend to relax into an uncurved (or less curved) state. For another example, during a complete copy of the inner rigidizing device, in which the outer rigidizing device is fully advanced over the inner rigidizing device, to advance the inner rigidizing device out of the outer device, the inner device must initially be driven straight out of the outer device before it can be articulated.

In some examples, the actuating steering member(s) (e.g., cables 7624) can provide a bending moment that is maintained at approximately the same bending moment during shape copying. Maintaining the bending moment can advantageously help the inner rigidizing device to hold its current shape during the copying process, improving shape copying fidelity. Maintaining the bending moment during shape copying can also reduce artificial ‘tightening’ of the bend as the exposed length of the inner rigidizing device is reduced. Additionally, maintaining the bending moment may allow for retaining/setting/resetting a desired curvature for the inner rigidizing device while it is positioned within the outer device. When the inner rigidizing device is subsequently advanced, it may advance along a constant curvature arc. This control can allow, for example, a user to drive the inner rigidizing device out along the tightest bend possible.

In some examples, the system is configured to automatically use the actuating steering member(s) as described above to aid in the transition of the inner device from the rigidized to the flexible state.

In some examples, the system can still allow the commanded curvature to be reset to zero upon completion of shape copies.

For example, the system can be configured to maintain an existing curvature command for partial shape copies, and to reset the curvature command to zero for full shape copies.

In some examples, the operator can select whether or not to reset the curvature to zero at the completion of a complete shape copy (e.g., partial shape copy, full shape copy, both partial and full shape copies).

In some examples, for a continuously-commanded shape copy, in which the shape copy progresses gradually while the operator activates a control (e.g., holds down a button) and stops when the operator releases the control (e.g., releases the button), the previously commanded curvature can be maintained during advancement of the outer rigidizing device 2400 and then gradually reduced to zero if the operator continues to activate the control after the outer device has advanced the full allowed extent.

The actuating steering member(s) may use two primary components to control the inner device distal tip bending section. For example, when a steering cable (or tendon or the like) is used, the first is by imparting a bending moment. A bending moment can be generated by stretching the steering cables. The second component is by imparting a geometric change. A geometric change can be imparted by displacing the steering cable, causing different path lengths along different steering cables, resulting in bends being formed. The effect of steering cable displacement depends upon the shape of the whole bending section, including the portion of the bending section, if any, positioned within the outer device.

As such, in some examples, the shape of the outer rigidized device may be used to control the shape of the inner rigidizing device as it transitions to a flexible state. The shape can be known using shape sensing technology. In some examples, tracking the movements of the inner rigidizing device can allow estimation of the copied shape of the outer device.

The shape of the inner device may generally be preserved during a shape copy. This can allow for a smooth exit from the shape copying sequence, because there is no change to the actuating steering member(s) control. It can still be important to know the distal shape of the outer device as the inner device advances, because less and less of the inner device distal tip will be subject to the shape constraint of the outer device as the inner device advances.

While an ideal shape copy would exactly preserve the shape of the copied device, in practice, the process can result in a slightly modified shape due to factors such as measurement error, physical effects such as different radial tolerancing between the devices, less or non-bendable sections of the copying device. To account for the modification in shape while estimating the shape of the copying device, the system can assume that the shape loses a certain amount or percentage (e.g., about 10%, 5-10%, 5-20%, etc.) of the current curvature. In such examples, the system may slightly modify the actuating steering member(s) controls before transitioning the copied inner device to a flexible state to maintain the curvature of the outer rigidized device.

Withdrawing the nested system can be a reversal of the forward sequence described above. The commanded curvature can be maintained or adjusted/controlled during withdrawal of the outer rigidized copying device.

A difference during withdrawal is that the actuating steering member(s) may not be able to provide sufficient degrees of freedom for the inner device to maintain the previous shape that it had within the outer device (e.g., when the shape comprises multiple curves in different directions). It has been found that maintaining or approximating the shape of the inner device along the portion of the inner device that will be exposed after retraction of the outer device can provide the smoothest transition for the inner device as it transitions out of a rigidized state.

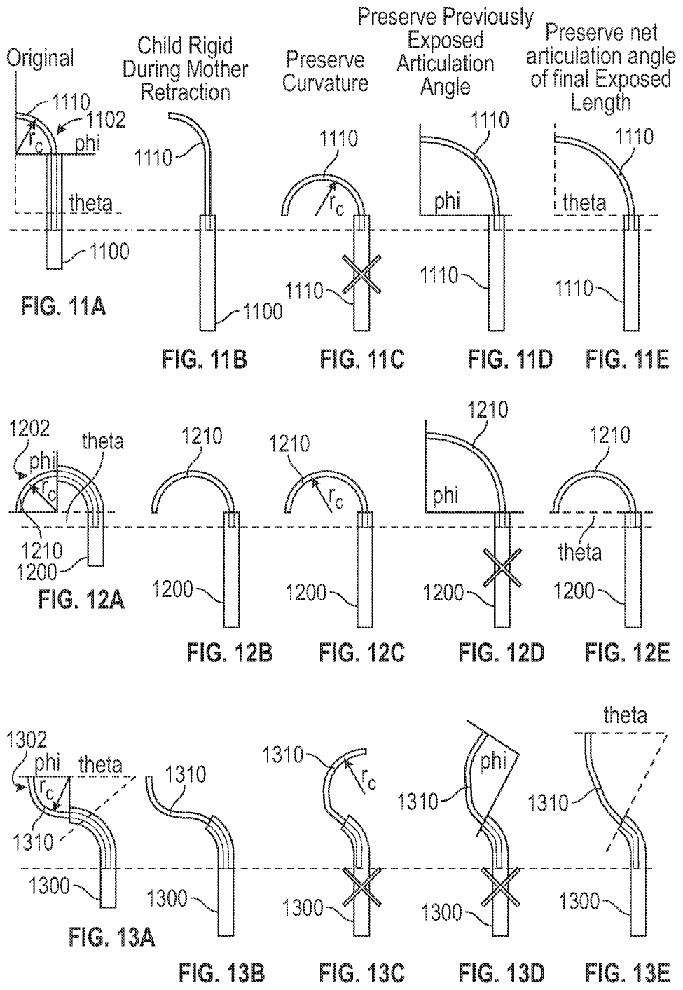

Different examples of controlling the copied device as it transitions to a flexible state after retracing an outer rigidizing device (“mother” or outer rigidizing member) relative to an inner rigidizing device (“child” or inner rigidizing member) are shown in FIGS. 11A-13E.

Referring to FIGS. 11A-11E, various examples of retracting an outer rigidizing device 1100 while actuating the actuating steering member(s) (e.g., steering cables) of the inner device to various degrees to adjust and/or preserve the shape of the exposed inner rigidizing device 1110 are shown. FIG. 11A shows the initial position of the inner rigidizing device 1110 and the outer rigidizing device 1100. In this example, the inner rigidizing device 1110 extends from the outer rigidizing device and curves in one direction and one plane. The exposed portion 1102 of the inner device is bent at an angle phi, and at a radius of curvature r_{c}. A portion of the bending section of the inner rigidizing device is within the outer rigidizing device in FIG. 11A. FIG. 11A also shows the net articulation angle, theta. This is the angle of the inner rigidizing device 1110 between the distal tip of the inner rigidizing device 1110 and a proximal point on the inner rigidizing device to which the outer device will be retracted. FIG. 11B shows the nested pair of devices 1100, 1110 as the outer rigidizing device 1100 is retracted while the inner rigidizing device remains rigid.

FIG. 11C illustrates an example of an undesirable state, indicated by the “X,” in which the steering cables are tensioned to preserve the original radius of curvature (r_{c}), resulting in the position shown, in which the inner rigidizing device 1110 moves and hooks at its distal end. Unless it is prevented by a structure within the body lumen, the tip of the inner rigidizing device 1110 may bend as shown, as the now flexible distal end region is driven by the tension from the cables to preserve the radius of curvature in the exposed distal end region. This may also turn the distal face of the inner rigidizing member 1110 in a different direction, which may reposition the direction the distal end of the inner rigidizing device is pointed towards, including any cameras or working channels. As mentioned, this may be undesirable within the body lumen. This change in shape may result because the child (inner rigidizing device 1110) transitions from a rigid configuration in FIG. 11B to a flexible configuration in FIG. 11C with a bending movement being applied by the steering cable(s) in the distal end region; the newly exposed bending section can be further actuated, as shown.

In some examples, as shown in FIG. 11D, the steering cables may instead be controlled to maintain the angle phi during retraction of the outer rigidizing device 1100. In FIG. 11D, the apparatus may apply sufficient (and may adjust) bending moment, e.g., tension, on one or more of the steering cables so that the exposed articulation angle (phi) is maintained while the inner rigidizing device 1110 is de-rigidized. In this example the distal face of the inner rigidizing device 1110 remains oriented in the same direction. In some examples the controller may coordinate the application of the bending moment on the rigidizing device by the steering cables to maintain the articulation angle (phi). Thus, in some examples the controller may dynamically adjust the steering cable(s) as the inner rigidizing member is de-rigidized, e.g., by releasing the positive or negative pressure rigidizing the inner rigidizing member.

In some examples, the apparatus may be configured to maintain the theta angle (e.g., the net articulation angle between the distal end face of the inner rigidizing device 1110 relative to the point on the inner rigidizing device to which the outer rigidizing device will be retracted). In the example shown in FIGS. 11A-11D, the theta angle is equivalent to the phi angle. Thus, FIG. 11E shows the position of the outer rigidizing device 1100 and the inner rigidizing device 1110, when the steering cables are controlled to maintain the theta angle during retraction of the outer device 1100. As shown in FIGS. 11D and 11E, in this case, preserving the previously exposed articulation angle phi is equivalent to controlling the net articulation angle of the final exposed length. When, as is typically the case here, the more proximal region of the device is relatively fixed (e.g., with respect to the patient anatomy), maintaining the net articulation angle (theta) will maintain the orientation of the distal end face of the inner rigidizing member with respect to the patient anatomy (e.g., the lumen).

FIGS. 12A-12E illustrate another example in which the phi and theta angles are not identical. In FIGS. 12A-12E, similar to FIGS. 11A-11E, the apparatus includes an inner rigidizing device 1210 (child) and an outer rigidizing device 1200 (mother). In FIG. 12A the inner rigidizing device 1210 has a radius of curvature (r_{c}) but has a larger phi angle. The outer rigidizing member is to be withdrawn to approximately the same relative position as shown in FIGS. 11A-11E, however the net articulation angle, theta is different (in this example, larger, e.g., 180 degrees) than the bend angle (phi) of the inner rigidizing device 1210 over the initial exposed region 1202. FIG. 12A shows the initial position of the inner rigidizing device 1210 and the outer rigidizing device 1200. In FIG. 12A, the inner rigidizing device 1210 extends distally from the outer rigidizing device and curves such that it hooks around to face proximally.

FIG. 12B shows the apparatus including the inner rigidizing device 1210 and the outer rigidizing device 1200 as the outer rigidizing device 1200 is retracted with the inner rigidizing device remaining rigid. The rigidity of the inner rigidizing device may then be released (e.g., by releasing the positive or negative pressure or otherwise) and the actuating steering member(s) (e.g., steering cables) may be used to preserve one or more of: the bend angle (phi), radius of curvature (r_{c}), and/or the net articulation angle (theta). For example, as shown in FIG. 12C the steering cables may be controlled by the controller (e.g., software, firmware, etc.) and used to preserve the original curvature r_{c}, results in the position shown in FIG. 12C, in which the inner rigidizing device 1210 remains hooked at its distal end as the outer rigidizing device is withdrawn proximally.

Alternatively, in some examples, the controller may maintain the same bend angle (phi) as shown in FIG. 12D. In this example, the controller may control (e.g., dynamically control) the actuating steering member(s) to maintain the angle phi during retraction of the outer device 1200. As shown by the “X” in this example, this may be a less desirable configuration, as the distal tip region may be translated as shown and may also change orientation and position of the distal face of device 1210.

In some examples the controller may instead maintain the net articulation angle (theta) and may maintain the approximate orientation (and in some examples the position) of the distal end face of the inner rigidizing device. In FIG. 12E the position of the inner rigidizing device 1210 may be maintained by the actuating steering member(s) (to maintain the angle theta) during retraction of the outer rigidizing device 1200. As shown in FIGS. 12C and 12E, in this example, maintaining the radius of curvature r_{c} and/or the net articulation angle (theta) of the inner rigidizing member by controlling bending moment of, e.g., the steering cables as the pressure (positive or negative) is released may result in the exposed length of the inner rigidizing device having essentially the same shapes, and approximately maintaining the position and orientation of the distal face of device 1210, particularly when controlled in simple configurations of the outer and inner rigidizing devices.

FIGS. 13A-13E illustrate an example in which the inner rigidizing member 1310 of the apparatus has a compound curvature (curving in more than one direction or plane) at the distal end region (e.g., the region that may be exposed by withdrawing the outer rigidizing member proximally) and illustrates the effect of controlling the shape during withdrawal of a rigidizing device by controlling either radius of curvature (r_{c}), bend angle (phi) or net articulation angle (theta). Once again, it may be most desirable to maintain the net articulation angle (theta) in order to preserve the position/orientation of the distal face of the inner rigidizing device 1310. FIG. 13A shows the apparatus with a distal radius of curvature (r_{c}) and bend angle (phi). In this example the bend angle is different than the net articulation angle (theta). In this example, the inner device 1310 extends distally from the outer device and curves in a first direction, similar to the exposed portion of the device 1110 in FIG. 11A. However, in this example, a more proximal portion of the inner rigidizing device 1310 (proximal to the exposed outer distal portion 1302) and the outer rigidizing device 1300 initially positioned over the inner rigidizing device 1310 curves in a second direction that is different from the first direction. The bend angle and radius of curvature of the second region may be the same or different from the bend angle and radius of curvature of the distal end region. Similarly, the bending plane and the bend angel may be different or the same. In this example, the net articulation angle is theta. FIG. 13B shows the inner rigidizing device 1310 still in a rigid configuration while the outer rigidizing device 1300 is withdrawn proximally. The apparatus, e.g., a controller, may then coordinate the bending moment applied by the actuating steering member(s) to maintain the same radius of curvature (r_{c}), bend angle (phi) or net articulation angle (theta) as the pressure (positive or negative) is released.

For example, in FIG. 13C, the controller may control the actuating steering member(s) to preserve the original radius of curvature r_{c}, resulting in the position shown in FIG. 13C, in which the distal end of the inner rigidizing device 1310 is in a different orientation and is significantly displaced relative to the original position when it was rigidized. As indicated by the “X” in this example, this may therefore be an undesirable outcome.

FIG. 13D illustrates an example in which the controller of the apparatus may instead maintain a constant bend angle (phi). In this example both the position of the distal tip of the inner rigidizing device 1310 and the orientation of the distal face of this distal tip are substantially different from the initial position (as shown in FIGS. 13A-13B). Thus this example is also marked with an “X” as shown.

In contrast, in FIG. 13E, the controller may coordinate the bending moment applied to the actuating steering member(s) to maintain the net articulation angle (theta) following retraction of the outer device 1300, and the release of rigidizing pressure (positive or negative) of the inner rigidizing device 1310. In FIG. 13E the curvature of the inner device 1310 is different from that of the original shape shown in FIGS. 13A and 13B, but the orientation of the distal face is maintained.

Thus, in any of these apparatuses, the controller may coordinate the actuating steering member(s) to maintain the net articulation angle (theta) and/or the orientation of the distal face of the inner rigidizing device during retraction of the outer rigidizing device. Preserving the net articulation angle, theta, may result in the mostly closely matched orientation and position of the distal face of the inner rigidizing device following release of the rigidization of the inner rigidizing device. This may allow a reasonably smooth transition between the rigidized and flexible states when retracting (and in some cases advancing) the apparatus. As illustrated by these examples, preserving the net articulation angle, theta, resulted in an maintaining the orientation of the distal face of the inner rigidizing device as the inner rigidizing device is transitioned from a rigidized state to a less rigid (e.g., flexible) state. Maintaining the net articulation angle (theta) or tip orientation may result in a shape of the device after retraction that approximates the shape before retraction.

As shown in FIGS. 11A-13E, the articulation angle, theta, may be measured between a plane including the distal face of the inner rigidizing device (e.g., device 1110, 1210, 1310, etc.) a cross section of the inner rigidizing device taken at the point to which the inner rigidizing device will be exposed (or at any point further proximal that is relative fixed in position with respect to the patient's anatomy). Measuring the articulation angle in this manner may help to preserve the orientational of the distal face of the inner rigidizing device (e.g., the direction it faces), which can be useful in maintaining the orientation (e.g., pointing direction) of the camera rather than its position, in applications in which the inner device comprises a camera. Maintaining the orientation of the camera can help to provide continuity and accuracy during imaging procedures.

It will be appreciated that the net articulation angle can be measured differently in a manner that still best preserves the shape of the inner device to be exposed, but without necessarily maintaining the angle of the distal end of the inner device.

As described above, the shape of the inner and outer devices can be known using shape sensing technology. In some examples, tracking the movements of the inner rigidizing device can allow estimation of the copied shape of the outer device.

In any of these examples, when transitioning the inner device from a rigidized to a flexible state the actuating steering member(s) may be adjusted before the device is de-rigidized. This sequence can allow a smoother and/or more predictable transition between the initial shape of the rigidized inner device and the subsequent shape of the child device in a flexible state.

In some examples, the actuating steering member(s) are adjusted as the outer rigidizing device is being advanced or retracted. This sequence can improve user responsiveness as the inner device is immediately ready for de-rigidization as soon as the copying process is complete.

### Methods for Screening of a Body Lumen

In some examples, the apparatus may be configured to be operable in a ‘screening mode’ in which the apparatus performs automated motions of the inner rigidizing device to sweep the camera view over surfaces of the inside of the lumen (e.g., intestine) to search for abnormalities (e.g., polyps). The automated motion may comprise a circling movement of the inner device. The inner rigidizing device may include one or more cameras at its distal end. In some examples the automated movement may be performed as the system retracts the apparatus through the lumen. In some examples the controller of the apparatus may include control logic to coordinate movement of the inner rigidizing device to scan in a screening mode as described herein.

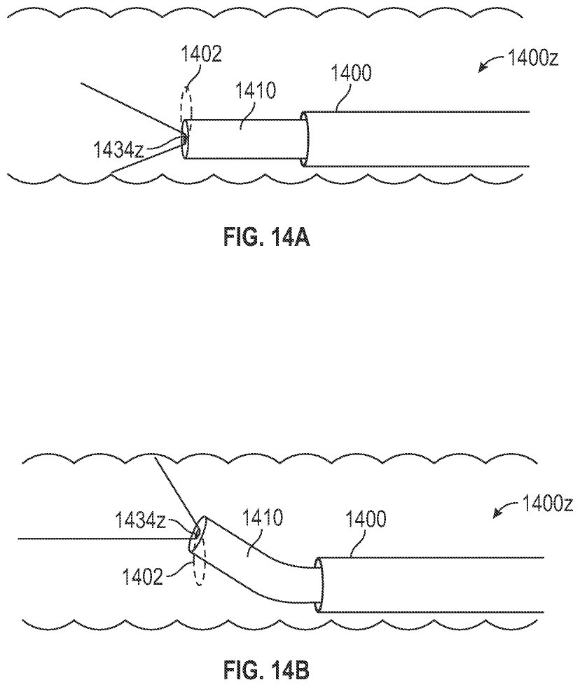

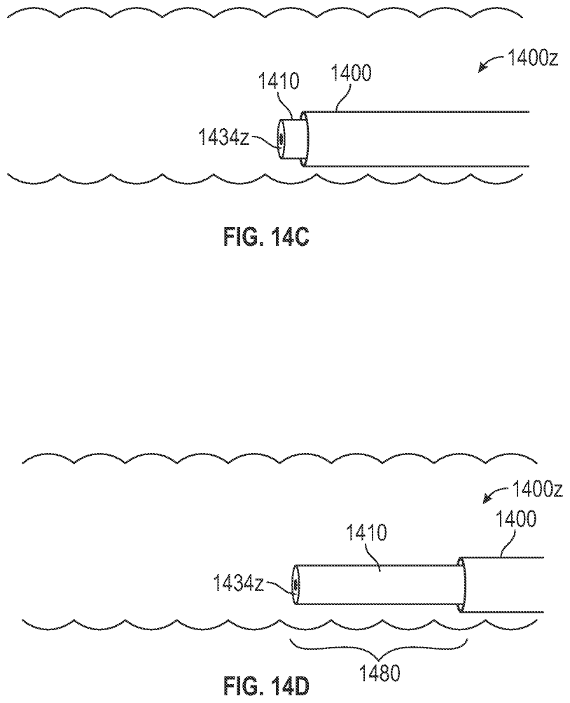

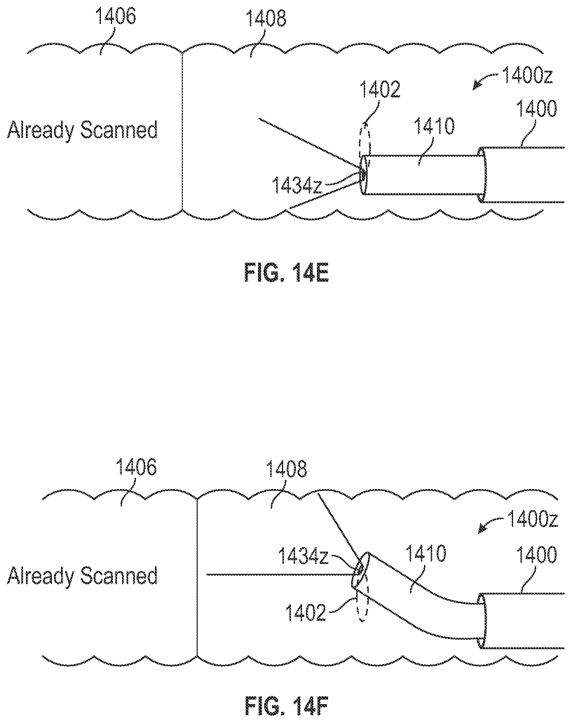

FIGS. 14A-14F schematically illustrate an example of an apparatus comprising an inner rigidizing device 1410 and an outer rigidizing device 1400. In one example of a screening mode, the apparatus 1400z can allow selection of an initial orientation for the camera 1434z positioned at a distal end of the inner rigidizing device. At this point, a user may select an orientation of the inner device such as orienting the camera view in the center of the lumen and indicating a center point (centered in loop 1402) within the lumen, as shown in FIG. 14A. The bending section of the inner device then rotates about that center point, in a circular pass movement 1402, to capture views of the walls all around and distal to the center point. FIG. 14B shows the inner rigidizing device 1410 at a different point during the rotation. The inner rigidizing device 1410 is shown in a flexible configuration (e.g., un-rigidized), while the outer rigidizing device 1400 is rigidized (e.g., by the application of positive or negative pressure). In some examples, the inner rigidizing device 1410 may be advanced distally and/or retracted slightly (e.g., distally/proximally) as it is navigated in a circular pass movement, in order to maintain the tip in a plane that is transverse to the region of the lumen where it is located. In any of these examples, the controller may automatically control the actuating steering member(s) and/or the rigidity of the inner rigidizing device and/or outer rigidizing device to coordinate the rotation and imaging. The controller may coordinate the rate of rotation and movement of the tip of the inner rigidizing member; in some examples the rate of rotation and movement may be based on the ability of an image collection and analysis module of the apparatus to collect and process images collected. The rate may be constant or may be variable. For example, the controller may adjust the rate of rotation and/or movement of the tip under automatic or semi-automatic control based on feedback from the image collection and/or processing (e.g., image analysis) module. In some examples this may allow the system to image the lumen more rapidly than manual review, but may allow for the user (e.g., physician, technical, etc.) to concurrently or later review the imaged lumen or a model (e.g., data file) of the lumen generated by the image collection and/or processing module.

Referring now to FIG. 14C, once the circular pass movement 1402 is complete, the apparatus 1400z may be operated to retract a selected length (or in some examples, advance a selected length). In the example shown in FIG. 14C, the inner rigidizing device 1410, in a flexible/non-rigid configuration, may be retracted into the outer rigidizing device 1400. In this example, the actuating steering member(s) (e.g., steering cables) may be allowed to adjust to relax or tension so that the inner rigidizing device may be readily drawn into the outer rigidizing device 1400. Although the example shown in FIGS. 14A-14D show the apparatus and lumen extending in a straight line, the procedure described herein may be performed in curved or tortuous lumen as well.

As shown in FIG. 14D, the outer rigidizing device 1400 may then be retracted relative to the inner rigidizing device 1410, to expose a section of the inner device 1410. In some examples the inner rigidizing device 1410 may be rigidized (e.g., by the application of positive or negative pressure, or otherwise) and the outer rigidizing device 1400 may be made flexible/de-rigidized (e.g., by releasing the pressure) so that it may maintain the shape as it extends proximally. The methods and apparatuses described herein may take advantage of any of the methods and procedures described above when retracting the apparatus. For example, the controller may maintain the net articulation angle by controlling the actuating steering member(s) following withdrawal of the outer rigidizing device 1400 and transitioning of the inner rigidizing device 1410 into a flexible configuration.

The length that the outer rigidizing device is withdrawn 1480 may be the same or may vary and may be determined based on how much of the lumen wall has been screened by the camera (which may be at least partially dependent on the field of view of the camera). In some examples, the withdrawal length 1480 may depend at least in part on how curved the lumen is. The apparatus 1400z may be configured to retract a selected withdrawal length from a first position that allows viewing of a first portion of the lumen, to a second position that allows viewing of a second portion of the lumen. These portions may be non- or minimally-overlapping; for example, the second portion may be proximal to the first portion. For example, the first portion of the lumen and the second portion of the lumen may be positioned adjacent to one another such that there is not overlap between the portions. In some examples the adjacent portions may overlap, so that the first portion of the lumen and the second portion of the lumen overlap by a selected amount (e.g., 0-5 mm, 0-10 mm, etc.), which may help ensure complete scans.

Once the inner rigidizing device 1410 is retracted and the outer rigidizing device 1400 is exposed, as shown in FIG. 14D, the apparatus 1400z is ready to perform another circular pass of imaging. This process may be repeated over a predetermined or selected (e.g., automatically or user-selected) length of the lumen. For example, referring to FIGS. 14E and 14F, once the apparatus 1400z has retracted the withdrawal length, the inner device 1410 may again rotate in a circular pass movement 1402 to image the walls of the lumen. FIGS. 14E and 14F show the first portion 1406 of the lumen that was previously scanned (e.g., in FIG. 14B) immediately adjacent to a second portion 1408 of the lumen, that is proximal to the first portion of the lumen. The camera 1434z is shown at different points along the circular pass movement 1402 in FIGS. 14E and 14F.

As mentioned, in some examples imaging analysis can be performed on the received imaging data to produce a model of the lumen. For example, any of the apparatuses described herein may include an image collection and/or analysis module (e.g., an image processing module). The controller may coordinate and/or receive input from an image processing module to repeat imaging (e.g. performing the loop) or repositioning the distal tip based on feedback from the image processing module. In any of these examples, machine learning can be used to provide information regarding the center of the lumen. This information can be used along with the imaging data to produce a model of the lumen. In general, the image processing module may include one or more machine learning agents to determine the position and/or orientation of the tip of the apparatus during scanning or to assist in collecting the scans, identifying targets (e.g., polyps) and/or guiding or steering the apparatus.

### EXAMPLES

As mentioned above, any of these apparatuses and methods may be configured to perform predetermined series of coordinated operations in order to enhance, simplify and/or speed up operation of a nested pair of rigidizing devices, which may be collectively referred to herein as a robotic system. These predetermined series or set of coordinated operations may include movements advancing and/or retracting, rigidizing/de-rigidizing (e.g., transitioning from a rigid state to a flexible state), and/or rolling one or both of the rigidizing devices of the nested pair of rigidizing devices. These set of operations may be specific to operation with a pair of nested (e.g., telescoping) devices, and the rigidizing devices may be any of the rigidizing devices described herein. These predetermined series or sets of coordinated operations may be automatically performed, based on one or more sensed or detected parameters (e.g., relative position of the nested rigidizing devices, sensed shape of one or more of the rigidizing devices, position relative to the patient's body, etc.) and/or may be activated by the user operating the apparatus.

For example, FIGS. 11A-11E, 12A-12E and 13A-13E, discussed above, illustrate one example of a method for controlling a nested pair of rigidizing devices 1100, 1110 so that the distal end face of one of rigidizing devices (e.g., the inner rigidizing device 1110) faces approximately the same direction when retracting the device. This may also be referred to as maintaining approximately the same net articulation angle between a distal end of the second rigidizing device with respect to a proximal portion of the second rigidizing device. As described, this may provide a smooth and clear image to the user when retracting the device, even through tortuous anatomy. For example, the method may be a method of controlling a nested pair of rigidizing devices comprising: retracting a first rigidizing device of the nested pair of rigidizing devices relative to a second rigidizing device of the nested pair of rigidizing devices, while the first rigidizing device is in a flexible state and the second rigidizing device is in a rigidized state; rigidizing the first rigidizing device; actuating steering members within the second rigidizing device to maintain a direction of a distal end face of the second rigidizing device constant relative to an external region before and/or while transitioning the second rigidizing device from the rigid state to the flexible state; and retracting the second rigidizing device relative to the first rigidizing device while the second rigidizing device is in the flexible state.

As mentioned, any of these methods may be implemented as an apparatus (e.g., system) including a nested pair of rigidizing devices comprising a first rigidizing device and a second rigidizing device; one or more processors; and a memory coupled to the one or more processors, the memory storing computer-program instructions, that, when executed by the one or more processors, perform the method (e.g., the computer-implemented method) for controlling the nested pair of rigidizing devices.

Any of these methods may include rigidizing and de-rigidizing (e.g., transitioning between the rigid state and the flexible state) by controlling pressure, e.g., positive and/or negative pressure. As mentioned above, the application of positive and/or negative pressure may apply or release the force driving a bladder layer against a rigidizing layer. In some examples the apparatus may include a source of positive and/or negative pressure and the controller may control and/or coordinate the application of positive and/or negative pressure to control the transition between the rigid state and the flexible state for each of the rigidizing devices of the nested pair of rigidizing devices.

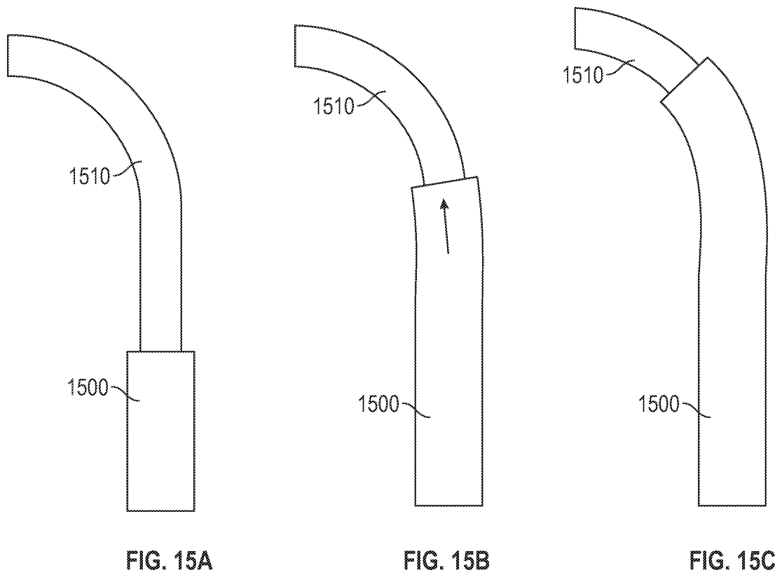

For example, FIGS. 15A-15C illustrate an example of a method of automatically shape copying between a pair of nested rigidizing devices, which may allow continuous copying of the shape of one rigidizing device relative to a second rigidizing device. This continuous copying may be triggered, for example, by a user actuating a control (e.g., button, dial, switch, pedal, etc.) to trigger shape copying. In some examples the method (or system performing the method) may be configured to continue the continuous and automatic shape copying only while the user continues to actuate the control (e.g., holds the button).

For example, in FIG. 15A the nested pair of rigidizing devices includes a first (e.g., outer) rigidizing device 1500 and a second (e.g., inner) rigidizing device 1510. The second rigidizing device may have just been extended distally and the tip region steered by controlling one or more actuating steering members (e.g., tendons, pull wires, etc.). In FIG. 15A, the user may then activate a control, e.g., push and hold a button, to trigger the copy command so that the first rigidizing device will copy the shape of the second (inner) rigidizing device, and in this example, the first rigidizing device begins to perform the copy sequence, as shown. In this example, the inner rigidizing device 1510 may optionally be rigidized (transitioned from the flexible state to the rigid state, e.g., by applying positive or negative pressure). The tension on the actuating steering members may be maintained at least until the inner rigidizing device is fully rigid. Similarly, the first (e.g., outer) rigidizing device may be in or transitioned to a flexible state (e.g., by releasing positive and/or negative pressure). In FIG. 15B the user keeps pressing copy command input (control), and the outer member continues performing the copy sequence; as shown the first rigidizing device 1500 advances distally over the second (inner) rigidizing device, which remains in the rigid state, while the outer rigidizing device 1500 remains in the flexible state. Each of these steps may be controlled and coordinated by the controller. At any point during this process if the user discontinues the control (e.g., releases the button), the shape copying may stop. For example, the user may release the copy command and the outer rigidizing device 1500 ends the copy sequence where it is, regardless of how much of the inner rigidizing device 1510 has been copied.

Thus, FIGS. 15A-15C illustrate a method of controlling a nested pair of rigidizing devices including: receiving a copy command from a user input; automatically performing a shape copying sequence, wherein the shape copying sequence comprises: advancing a first rigidizing device of the nested pair of rigidizing devices relative to a second rigidizing device of the nested pair of rigidizing devices, while the first rigidizing device is in a flexible state and the second rigidizing device is in a rigidized state, wherein the first rigidizing device is initially proximal to the second rigidizing device so that first rigidizing device copies the shape of the second rigidizing device; and preventing the first rigidizing device from advancing distal to the second rigidizing device.

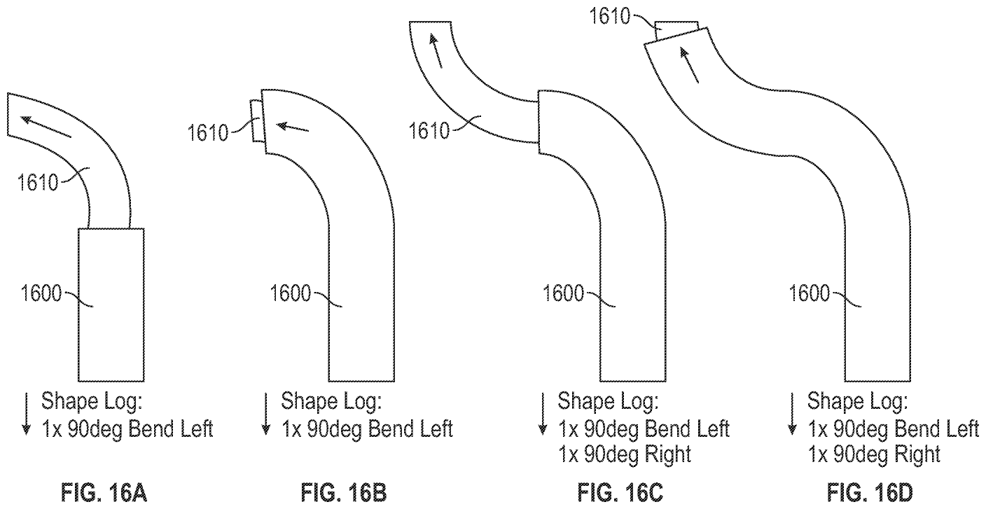

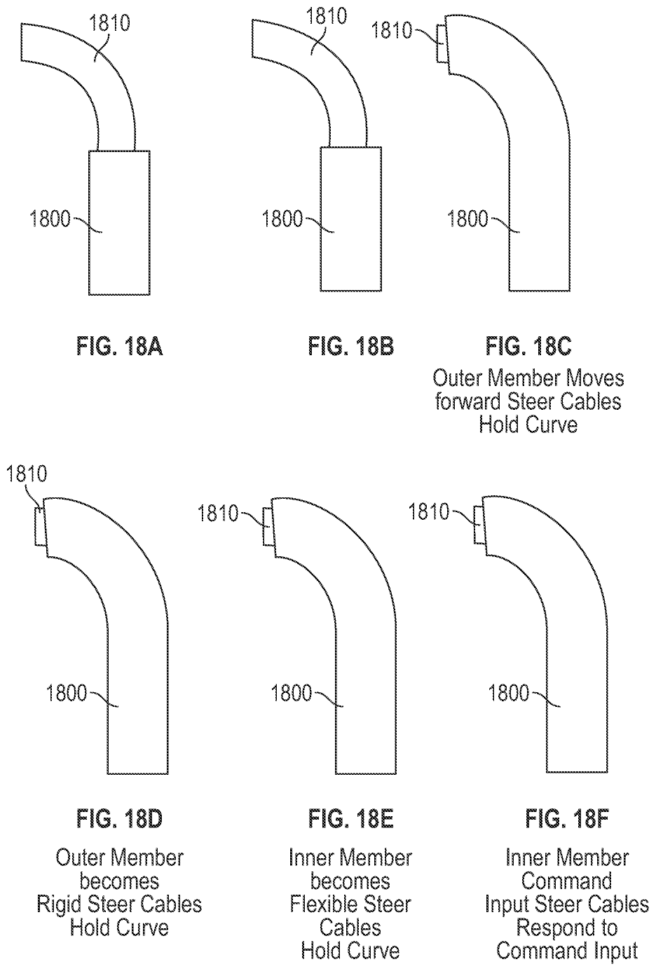

Also described herein are predetermined series of coordinated operations for shape copying when advancing and/or when retracting the apparatus. In general, these methods and apparatuses may perform multiple, repeated steps of shape copying. Any of these examples may include tracking or remembering the shape. For example, the methods or apparatuses may include tracking the angle of the bending section (e.g., distal tip region) of the inner rigidizing device that is nested in the outer rigidizing device. FIGS. 16A-16D illustrate multiple shape copying steps when advancing a nested pair of rigidizing devices. In FIG. 16A the inner rigidizing device 1610 is extending distally from the outer rigidizing device 1600, and may have been steered, using one or more actuating steering members into a curved shape, while being advanced distally out of the outer rigidizing device 1600. The inner rigidizing device is shown bent at an approximately 90 degree angle, bending to the left. The outer rigidizing device may copy this shape, as shown in FIG. 16B by advancing distally over the inner rigidizing device when the inner rigidizing device has been set to the rigid state and the outer rigidizing device has been set to the flexible state. Once the shape of the inner rigidizing device has been copied by the outer rigidizing device, the outer rigidizing device may be rigidized, e.g., transformed into the rigid state, and the inner rigidizing device may be transitioned to the flexible state so that it can be advanced distally while being steered, e.g., by actuating of one or more actuation steering members, as shown in FIG. 16C. The apparatus may control the tension applied to the actuating steering members, as described in more detail in reference to FIGS. 18A-18F, below. In FIG. 16D the shape copying described in FIGS. 16A-16B may be repeated, so that the outer rigidizing device 1600 may be advanced in the flexible state over the inner rigidizing device 1610 in the rigid state. In general, these repeated steps of shape copying described in FIGS. 16A-16D may be automated or assisted by the controller of the system, e.g., using automated shape copying (e.g., similar to FIG. 15A-15C), and or generally coordinating the transitions between rigid and flexible states of the outer rigidizing device and inner rigidizing device.

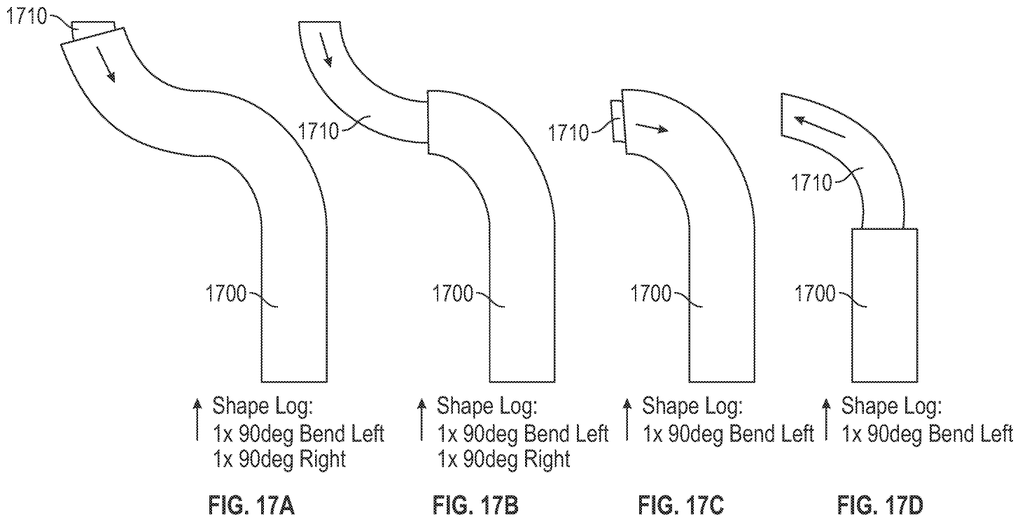

The method described in FIGS. 16A-16D illustrate an example of the apparatuses and methods described herein remembering the shapes that were previously copied proximal to the articulating section of the inner member, and that this memory, preserved in the shaped of the nested region, allows the inner member to be reversed, e.g., backwards into the existing rigidized outer member shape when retracting the system backwards, as described in FIGS. 17A-17D, below. In FIGS. 16A-16D, the method illustrates the process of inchworming the forward and preserving the shapes (history) of curves that the system has copied. As described for FIGS. 17A-17D, below, these methods may allow reversing through those same curves that were preserved when advancing distally, using the actuating steering member(s) (e.g., steering cables) to gradually bend the articulating distal end region into that previously remembered shape each time in order to better preserve the proximal shape of the system, since it may otherwise drive the previous curves around while retracting backwards if the steerable region did not at least somewhat approximate the shape of that section; the bending section may otherwise default to a less bent (or un-bent) articulation as it retracts backwards into the outer member.

This remembering or preserving of the curvature therefore allows tracking of the entire rigidizing length, not just the part that is being actively steered, without having to directly sense that shape. As described in reference to FIGS. 11A-11E, 12A-12E and 13A-132E since the shape of the outer member just proximal to the articulating section is preserved (and “known”) by these apparatuses. Thus, the curves that have been laid down and copied may be preserved as a picture of what shape the total system is in based on the articulation of the steerable distal end region, without requiring direct sensing of the shape of those curves.

FIGS. 17A-17C illustrate an example of shape copying when retracing the apparatus. In general, when reversing or retracting (proximally) the apparatus, the inner rigidizing device may shape copy the outer rigidizing device, whereas when advancing (distally) may involve the outer rigidizing device shape copying the inner rigidizing device, as described in FIGS. 16A-16D. In FIG. 17A, the apparatus includes an outer rigidizing device 1700 and an inner rigidizing device 1710 that are nested together. FIG. 17A shows the nested pair of rigidizing devices in which the inner rigidizing device 1710 has been retracted, in a flexible state, into the first rigidizing device 1700 which is held in the rigid state. Although in some cases it may be possible to withdraw the entire apparatus from the patient by transitioning both the inner rigidizing device and the outer rigidizing device to a flexible state (and relaxing the steering member(s)), in many cases it would be more preferable to retract the device from the distal end regions of the apparatus, leaving the more proximal curves and bends, which may prevent strain on the luminal walls and may allow controllable visualization of the surrounding region as the device is withdrawn. For example, as shown in FIGS. 17B-17C, the apparatus may be smoothly withdrawn proximally over the various curves by maintaining the inner rigidizing device 1710 in a rigid state, preferably with the articulating steering member under tension, holding the curvature of the distal tip; the outer rigidizing device may be in the flexible state and may be withdrawn proximally over the distal most bend in the inner rigidizing device, as shown. The outer rigidizing device 1700 may then be rigidized and the inner rigidizing device 1710 may be transitioned to the flexible state and retracted proximally into the now rigid outer rigidizing device 1700. These steps may be repeated as shown in FIGS. 17C-17D.

In any of these steps it may be helpful (and the system may be configured) to steer the distal tip region of the inner rigidizing device as the inner rigidizing device is withdrawn proximally into the outer rigidizing device. This may be achieved as described above, e.g., be maintaining an approximately constant direction of the face and/or the maintaining a net articulation angle between a distal end of the second rigidizing device with respect to a proximal portion of the second rigidizing device.

FIGS. 18A-18F illustrate one example of a method of controlling the timing of the release of tension on the distal tip of the inner rigidizing device, which may be steerable at its distal tip region, when transitioning from rigid to flexible state by actuating steering members to maintain a curvature as the device transitions to the flexible state. Thus, FIGS. 18A-18F illustrates the timing of the transition of driving of the inner 1810 and/or outer 1800 rigidizing device after making a copy.

For example, FIGS. 18A-18F illustrate a method of controlling a nested pair of rigidizing devices by: advancing the first rigidizing device 1800 of the nested pair of rigidizing devices distally relative to a second rigidizing device 1810 of the nested pair of rigidizing devices, wherein the first rigidizing device 1800 is in a flexible state and the second rigidizing device 1810 is in a rigid state; transitioning the first rigidizing device 1800 from the flexible state to the rigid state; and transitioning the second rigidizing device from the rigid state to the flexible state while actuating steering members of the second rigidizing device to maintain a curvature of a distal end of the second rigidizing device as the second rigidizing device transitions to the flexible state.

In FIG. 18A the inner (e.g., second in this example) rigidizing device creates a shape (curve), and the actuating steering members may hold the shape. For example, the actuating steering members may be steel cables. In FIG. 18B, the inner rigidizing device 1810 may then be transitioned to the rigid state; the actuating steering members may hold the bend formed by the distal end region of the inner rigidizing device. In FIG. 18C, the outer rigidizing device 1800 may then advance, in the flexible state, over the inner rigidizing member while the inner rigidizing member remains in the rigid state and the actuating steering members hold the bend in the steerable tip region. In FIG. 18D, the outer rigidizing device may then be rigidized (e.g., by applying pressure) to the rigid state, while the actuating steering members hold the bend in the steerable tip region in the inner rigidizing member. In FIG. 18E the inner member may then be transitioned to the flexible state (e.g., by releasing the pressure on the inner rigidizing member), while the actuating steering members hold the bend in the steerable tip region. Finally, in FIG. 18F the inner member may be actively steered, by controlling the actuating steering members, which may respond to command input.

Any of the methods and apparatuses described herein may be configured so that one of the rigidizing devices automatically (or semi-automatically, e.g., after confirming with the user, or after the user triggers an automatic mode, etc.) copies the shape of the other rigidizing device when an automatic copying trigger event is detected (e.g., when the automatic copying trigger threshold is detected). For example, the automatic copy trigger event may be when the distance between the distal end of the two rigidizing devices exceeds a threshold, when the user stops motion of one (e.g., the inner) rigidizing devices, when the driving performance of the first (e.g., outer) and/or second (e.g., inner) rigidizing device has degraded below a threshold, e.g., based on feedback from the camera and/or a shape sensor following a movement command, or some other heuristic.

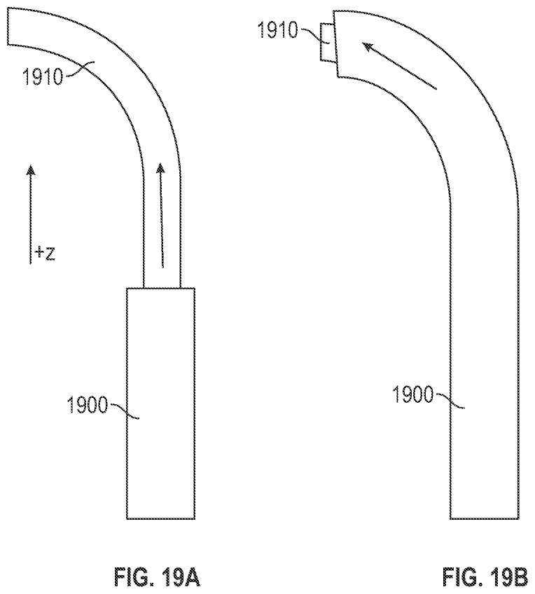

For example, in some examples the apparatus and method may be configured so that one of the rigidizing devices automatically copies the shape of the other rigidizing device when the distance between the distal end of the two rigidizing devices exceeds a threshold. Thus, the automatic copying trigger threshold may be a relative axial travel distance between the first rigidizing device and the second rigidizing device. For example, automatic shape copying may be triggered when the distance between the distal end of the inner rigidizing device and the distal end of the outer rigidizing device is at a maximum distance of extension (in the “Z” direction), as shown in FIG. 19A. In this example, the method or apparatus may automatically make the outer rigidizing device 1900 copy the shape of the inner rigidizing device 1910. For example, the system may automatically perform the shape copying steps without requiring any input from the user. In FIG. 19B the inner rigidizing device 1910 may be maintained in the rigid state while the outer rigidizing device is in the flexible state and is advanced distally over the inner rigidizing device.

FIGS. 19A-19B illustrates a method of controlling a nested pair of rigidizing devices by: advancing a second rigidizing device of the nested pair of rigidizing devices distally relative to a first rigidizing device of the nested pair of rigidizing devices, while the second rigidizing device is in a flexible state and the first rigidizing device is in a rigidized state; and automatically performing a shape copying sequence when the second rigidizing member extends to a predetermined travel distance relative to the first rigidizing member, wherein the shape copying sequence comprises: advancing the first rigidizing device relative to the second rigidizing device, while the first rigidizing device is in a flexible state and the second rigidizing device is in a rigidized state.

An of the automatic copying trigger events causing one of the rigidizing devices to automatically or semi-automatically copy the shape of the other rigidizing device may be controlled by the controller, which may receive input (e.g., sensor input) and may apply control logic to drive automatic or semi-automatic movement of the rigidizing device.

For example, the automatic copying trigger event may be stopping of movement of one or both of the first and second rigidizing devices. Thus, the automatic copying trigger threshold may be a time delay (e.g., a copying trigger event time delay) following movement of the second rigidizing member relative to the first rigidizing member. In one example, the apparatus may be configured to detect stopping of movement of a steerable inner rigidizing device; movement of the inner rigidizing device may be detected by user input advancing and/or retracting the inner rigidizing device, and/or by the user steering (bending) the inner rigidizing device. The automatic copying trigger event may be triggered when the controller does not detect a user input (e.g., advancing/retracting and/or steering) following a time delay. In some examples the apparatus may be configured (or controlled by user input to enter an automatic or semi-automatic state) so that after the steerable inner rigidizing device is advanced (either at all or by some minimum amount) so that the other rigidizing device is proximal to the steerable inner rigidizing device, once the controller no longer detects control input driving movement of the inner rigidizing device, e.g., steering and advancement (and optionally the controller also does not detect movement of the outer rigidizing device), after a time delay period (e.g., an automatic copying trigger event time delay) the controller may cause the outer rigidizing device to shape copy the inner rigidizing device, as described herein. The automatic copying trigger event time delay may a fixed time delay, e.g., 2 seconds or more, 3 seconds or more, 4 seconds or more, 5 seconds or more, 6 seconds or more, 7 seconds or more, 8 seconds or more, 9 seconds or more, 10 seconds or more, etc. Alternatively, the automatic copying trigger event time delay may be determined based on user input (preference) and/or based on prior movement commands, including steering and/or advancement/retraction. The shape copying may include rigidizing the inner rigidizing device (converting to a rigid state while controlling the articulating steering member(s) as described herein), de-rigidizing the outer rigidizing device (e.g., converting to a flexible state), and advancing the outer rigidizing device so that it extends fully or partially to the distal end region of the inner rigidizing device.

The controller may receive input from one so more sensors (e.g., sensor data) and or input from one or more controllers (e.g., control to advance/retract and/or steer). The control input and sensor data may be processed by the controller using control logic as described herein and used to identify one or more automatic copying trigger events. Another automatic copying trigger event may be coupled with analysis by the controller of driven movements, such as detection of movement performance; as mentioned, one automatic copying trigger event may be detection of degradation of driven movement of the inner and/or outer rigidizing device. Degradation of movement may be determined (and may trigger an automatic copying trigger event) when, for example, the inner and/or or outer rigidizing devices are slower in executing a movement command (advance/retracting, roll, and/or steering) than a threshold rate value. Alternatively or additionally, in some examples degradation of movement may be determined (and may trigger an automatic copying trigger event) when driving movement of the inner and/or outer rigidizing device does not perform a full or complete movement, e.g., an “expected” movement, based on position or shape detection (e.g., shape sensing), in some examples, based upon camera feedback vs commanded pose).

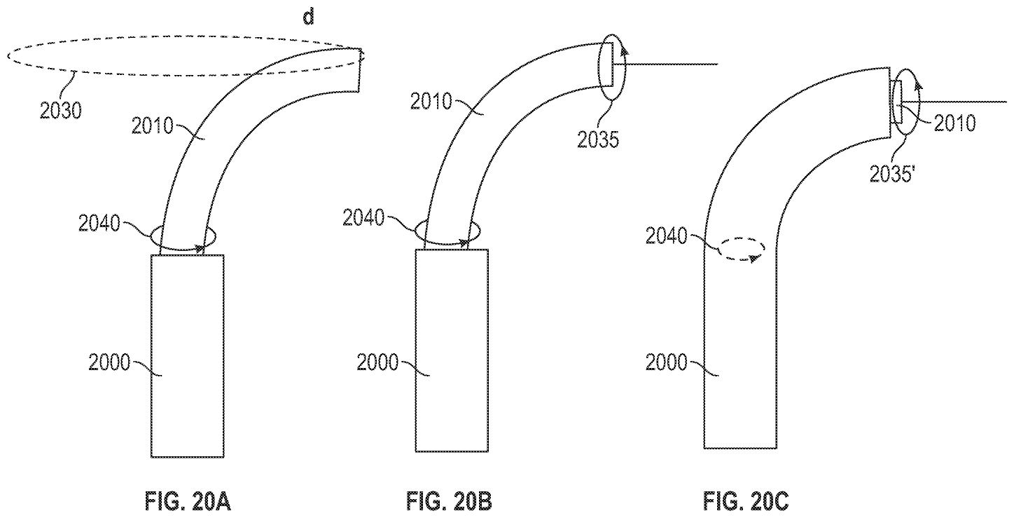

In any of these methods and apparatuses, the method may include moving the inner rigidizing device of the nested pair of rigidizing devices to pan around itself, e.g., to image the walls of a lumen in 360 degrees or near 360 degrees. For example, FIGS. 20A-20C illustrate different types of roll that may be coordinated by the methods and systems described herein. FIG. 20A illustrates rotation, e.g., roll, 2040 of the distal tip of the inner rigidizing device 2010 relative to the distal end of the outer rigidizing device 2000. This first type of roll of the inner rigidizing device 2040 results in the region of the distal tip that is bent and extending from the outer rigidizing device swinging in a loop 2030. This may be useful for panning around a structure (e.g., lumen). The inner rigidizing device 2010 may be in a rigid state or a flexible state. FIG. 20B illustrates the use of the actuating steering members to maintain the angle and orientation of the angle of the inner rigidizing device 2010 as the inner rigidizing device is rolled 2040. As a result, the distal face of the inner rigidizing device rotates (rolls) 2035 as shown. The inner rigidizing device may be in a flexible state in this example. This may be particularly useful in orienting the field of view of an imaging device forming the distal end region of the inner rigidizing device. A similar effect may be achieved by rigidizing the outer rigidizing device 2000 so that it may support roll of the inner rigidizing device, which may be in a flexible state, so that rolling the inner rigidizing device 2010 results in rolling of just the face of the apparatus.

Thus, for example if a user wants to rotate their view, a commanded rotation of the inner member 2010 may cause the user perspective to rotate about the centerline of the proximal-most face of the outer member due to the rigid support that it provides. If the inner member is articulated (FIG. 20B), this may cause the user viewpoint to travel along path d 2030. In order to instead rotate about the centerline of the inner member, either the system may command the articulating steering members (e.g., cables) to coordinate with the roll movement and keep the user facing the same direction, or, preferably, the outer member will copy to become flush with the inner member and support the rotation, so the user perspective now looks in the same direction but now only rotates the view, as shown in FIG. 20C.

Thus, the method or apparatus may be configured to achieve roll of the inner rigidizing device when examining a lesion or other scenario by performing a full shape copy in order to have the outer rigidizing device hold the inner rigidizing device in place while rolling, eliminating the need to use the actuating steering member(s) to coordinate the roll while the device is bent at an angle. This may allow the distal face of the inner device to roll about its centerline, instead of rotating the entire bending section, as shown in FIG. 20C.

In general, any of these apparatuses and methods may be configured to automatically or semi-automatically operate, and/or determine when the outer rigidizing device should perform a shape copy of the inner rigidizing device (or vice versa), and to what extent. For example, as described above in reference to FIG. 19, the apparatus may be configured to automatically performing a copy if the user reaches the end of their allowed insertion length of the inner rigidizing device.

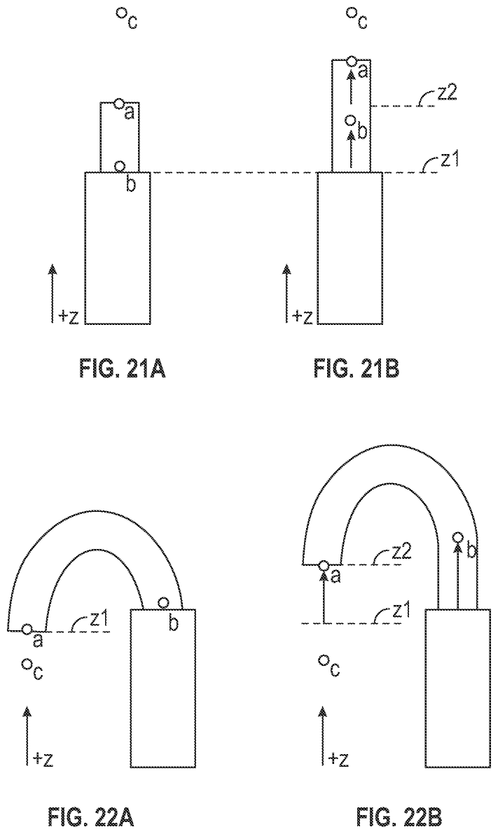

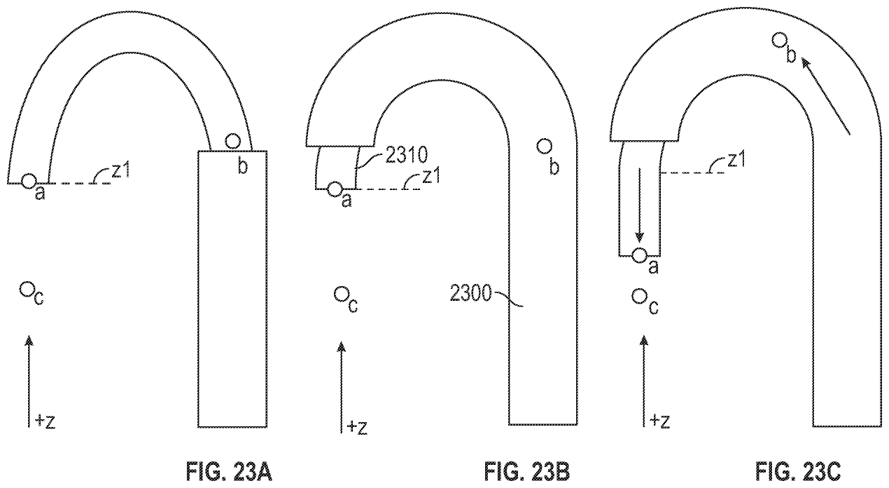

For example, in some situations it may be beneficial if the system detected a paradoxical motion of the device, to choose to automatically perform a shape copy in order to support the inner device motion in the new direction. This is illustrated, e.g., in FIGS. 21A-21B (showing a normal scenario), FIGS. 22A-22B (showing a paradoxical movement), and FIGS. 23A-23B (showing detection and correction for paradoxical movement).

In FIGS. 21A-21B, the inner rigidizing device is facing in the +z direction and there is no articulation in the inner rigidizing device. The user wants to reach point c, so inputs a command to move the system to advance forward. This moves both point b and point a (e.g., the distal face of the inner rigidizing member, which may correspond to the camera position) in the +z direction, and the user perspective at point a sees movement in the correct direction towards their goal.

However, in some situations, shown in FIGS. 22A-22B, the inner rigidizing device may be facing in the −z direction and there is close to about 180 degrees of articulation on the inner rigidizing device. The user wants to reach point c, so inputs a command to move the system forward. Because of the bend in the inner rigidizing device, this moves both point b and point a in the +z direction, but now (paradoxically) the user perspective at point a sees movement away from their goal of point c, despite inputting the same command and seeing what appears to be the same distance from point a to point c as in FIGS. 21A-21B. To the user, the system appears to be moving in the opposite direction than they have commanded.

Any of the apparatuses described herein may be configured to perform a method as described in FIGS. 22A-22B to correct and/or prevent paradoxical movement. In FIGS. 23A-23C, the inner rigidizing device is facing in the −z direction and there is close to 180 degrees of articulation on the inner member. The user wants to reach point c, so inputs a command to move the system forward. The system may be configured to recognize (e.g., through analyzing the change in distance from point c from the image or from recognizing the angle of articulation and the direction of motion that will occur due to it, and/or identifying the curvature of the rigidizing device and knowing where element 2300 is) that the user will not move in the desired direction, and therefore may automatically perform a shape copy to advance the outer rigidizing device to include all of the bends of the inner rigidizing device prior to advancing the inner rigidizing device; alternatively the apparatus may suggest that the user perform this shape copy. As illustrated in FIG. 23B the shape copy may include advancing the outer rigidizing device 2300 into the current position of the distal end region of the inner rigidizing device 2310. After performing the shape copy, if the user now inputs a command to move the system forward (as shown in FIG. 23C), the inner rigidizing device will slide along the rigid support from the outer rigidizing device, and point a will move in the −z direction towards the goal of point c, and the user's perspective will now move towards their goal of reaching point c.

In general, it should be recognized that although many of the examples described herein are described in the context of the use of these methods an apparatuses within a human (or other mammalian) body, any of these methods and apparatuses may be used within non-biological regions, such as, but not limited to, within ducts, pipes, geotechnical, or other regions.

### Single Full-Length Rigidizing Device Apparatuses

Although the majority of the apparatuses and methods described herein are described in the context of a nested pair of rigidizing devices in which both the inner and outer rigidizing devices have a relatively long lengths (longitudinal lengths) that are both rigidizing, many of these methods and apparatuses may be performed with a single rigidizing device having a relatively long rigidizable length and a non-rigidizing, or partially rigidizing, steerable device. The second, non-rigidizing or partially rigidizing steerable device may be rigidizing (or at least configured to control or lock the steered distal end region in selected curve or shape) over a length of the device that is shorter than the length of the rigidizing device having a relatively long rigidizable length. In some examples the locking or rigidizing length of the second device may be the length of just the steerable distal end region.

In particular, also described herein are methods and apparatuses including a first rigidizing device as described herein, which may be rigidized along the majority of its length (e.g., over half of its length, over 60% or its length, over 70% of its length, over 80% of its length, over 85% of its length, over 90% of its length, etc.) and a second steerable device in which the distal end region is steerable by one or more actuating steering members. This distal end region of the second device may be either rigidizing or pseudo-rigidizing (e.g., locking), but this distal end region may be much shorter than the rigidizing length of the first rigidizing device. For example, a second rigidizing device may be pseudo-rigidizing by controlling the actuating steering members so that the shape of the steerable distal end region is held (“locked”) in the determined or selected shape, while the region proximal to the steerable distal end region remains flexible. The first rigidizing device may be rigidizing along the majority of the inserted length, or at least a region that is longer than the steerable end region of the second device (e.g., 1.5× as long or longer, 2× as long or longer, 3× as long or longer, 4× as long or longer, 5× as long or longer 10× as long or longer, etc.). In some examples the first rigidizing device is an outer rigidizing device configured as described above. The second (steerable device) may be nested within the outer rigidizing device and may include the steerable distal end region that may be rigidized (or equivalently pseudo-rigidized by manipulating the actuating steerable member(s)).

Thus, in some examples the distal tip region of the inner member may therefore have no rigidizing structure but may be configured to hold a position using the actuating steering members (e.g., steering cables). Thus, the methods and apparatuses described herein may be performed by a nested pair of devices including a first, full-length, rigidizing device, and a second elongate device nested with the first, full-length, rigidizing device in which the second elongate device includes a steerable distal end region that may be locked in a selected shape. The selected shape may be a user-defined (steered) shape that may be copied by the first, full-length, rigidizing device, and/or it may be set by copying the curvature of a region of the first, full-length, rigidizing device. This selected shape may be held (“rigidized” or “pseudo-rigidized”) by controlling the actuating steering members. In some examples the selected shape may be held by locking a rigidizing layer as described above (“rigidized”). In some examples the steerable distal end region of the second elongate device may be controlled by actuating one or more actuating steering members; alternatively or additionally, the second elongate device may be rigidizable over just the distal end region including all or some of the steerable distal end region. In examples in which the second elongate device is rigidizing, the region that is rigidizing may be rigidizable over a much shorter length than the rigidizing region of the other (e.g., the first) rigidizing device.

For example, a method of controlling a nested pair of devices (including at least one rigidizing device configured to be rigidized along the majority of its length and a second rigidizing device configured to be rigidized along a distal steerable region of its length), may include: retracting a first rigidizing device of the nested pair of devices relative to a second device of the nested pair of devices, while the first rigidizing device is in a flexible state and the distal end region of the second rigidizing device is in a rigidized (e.g., locked) state; rigidizing the first rigidizing device; and actuating steering members within the second device to maintain a direction of a distal end face of the second device constant relative to an external region before and/or while transitioning the second device from the rigid state to the flexible state.

A method of controlling a nested pair of devices (including at least one rigidizing device configured to be rigidized along the majority of its length and a second rigidizing device configured to be rigidized along a distal steerable region of its length) may include: receiving a copy command from a user input; automatically performing a shape copying sequence, wherein the shape copying sequence comprises: advancing a first rigidizing device of the nested pair of devices relative to a second device of the nested pair of rigidizing devices, while the first device is in a flexible state and the second device is in a rigidized state in which the steerable distal end region is rigid, wherein the first rigidizing device is initially proximal to the second device so that first rigidizing device copies the shape of the second device; and optionally preventing the first rigidizing device from advancing distal to the second device. Advancing the first rigidizing device may include advancing the first rigidizing device only while the copy command is continuously received.

A method of controlling a nested pair of devices (including at least one rigidizing device configured to be rigidized along the majority of its length and a second rigidizing device configured to be rigidized along a distal steerable region of its length), the method comprising automatically performing a shape copying sequence when an automatic copying trigger event is detected by a control circuitry, may include: receiving, in the controller, one or more of sensor data and/or user movement input; comparing the one or more of sensor data and/or user movement input to an automatic copying trigger threshold; and triggering the shape copying sequence when the automatic copying trigger threshold is detected, wherein the shape copying sequence comprises: advancing the first rigidizing device relative to the second device, while the first rigidizing device is in a flexible state and the second device is in a rigidized state.

For example, a method of controlling a nested pair of rigidizing devices (including at least one rigidizing device configured to be rigidized along the majority of its length and a second rigidizing device configured to be rigidized along a distal steerable region of its length), may include: advancing a second rigidizing device of the nested pair of rigidizing devices distally relative to a first rigidizing device of the nested pair of rigidizing devices, while the second rigidizing device is in a flexible state and the first rigidizing device is in a rigidized state; and automatically performing a shape copying sequence when the second rigidizing member extends to a predetermined travel distance relative to the first rigidizing member, wherein the shape copying sequence comprises: advancing the first rigidizing device relative to the second rigidizing device, while the first rigidizing device is in a flexible state and the second rigidizing device is in a rigidized state.

A method of controlling a nested pair of rigidizing devices (including at least one rigidizing device configured to be rigidized along the majority of its length and a second rigidizing device configured to be rigidized along a distal steerable region of its length), may include: advancing a first rigidizing device of the nested pair of devices distally relative to a second device of the nested pair of devices, wherein the first rigidizing device is in a flexible state and the second device is in a rigid state in which the distal end region is rigid (e.g., locked); transitioning the first rigidizing device from the flexible state to the rigid state; and transitioning the second device from the rigid state to a flexible state (unlocking the steerable distal end region so that it may bend freely) by slowly releasing the steering members of the second device to release a curvature of a distal end of the second rigidizing device as the second device transitions to the flexible state.

A method of advancing or retracting a system comprising a nested pair of devices (including at least one rigidizing device configured to be rigidized along the majority of its length and a second rigidizing device configured to be rigidized along a distal steerable region of its length) along a body lumen may include: advancing or retracting a first device in a flexible state relative (in which the steerable distal end of the first device is allowed to move freely) to a second rigidizable device in a rigid state and steering the first device using steering members of the first device; rigidizing (e.g., locking) the first device so that the steerable distal end region is locked in a selected configuration; advancing or retracting the second rigidizing device in a flexible state at least partially over the rigidized first device; rigidizing the second rigidizing device; actuating the steering members to correspond to a curvature of the rigidized second rigidizing device; and transitioning the first device to a flexible state (e.g., by releasing tension on the steering members).

Similarly, a method of screening a body lumen of a patient may include: navigating a system, comprising a nested pair of devices including a first device positioned nested within a second rigidizing device, through the body lumen, the first device comprising a camera at a distal end, wherein the distal end region is steerable; exposing a distal portion of the first device; articulating the distal portion of the first device to perform a circular pass movement (e.g., loop) resulting in visualization by the camera of a circumference of a first portion of the body lumen; retracting the system by a selected length such that the distal portion of the first device is exposed; and articulating the distal portion of the first device to perform a circular pass movement resulting in visualization by the camera of a circumference of a second portion of the body lumen, wherein at least a portion of the second portion is positioned proximally to the first portion.

Also described are systems including any of these methods.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one example, the features and elements so described or shown can apply to other examples. It will also be appreciated by those of skill in the art that references to a structure or feature that is disposed “adjacent” another feature may have portions that overlap or underlie the adjacent feature.

Terminology used herein is for the purpose of describing particular examples only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units is also disclosed. For example, if 10 and 15 are disclosed, then 11, 12, 13, and 14 are also disclosed.

Although various illustrative examples are described above, any of a number of changes may be made to various examples without departing from the scope of the invention as described by the claims. For example, the order in which various described method steps are performed may often be changed in alternative examples, and in other alternative examples one or more method steps may be skipped altogether. Optional features of various device and system examples may be included in some examples and not in others. Therefore, the foregoing description is provided primarily for exemplary purposes and should not be interpreted to limit the scope of the invention as it is set forth in the claims.

The examples and illustrations included herein show, by way of illustration and not of limitation, specific examples in which the subject matter may be practiced. As mentioned, other examples may be utilized and derived there from, such that structural and logical substitutions and changes may be made without departing from the scope of this disclosure. Such examples of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1.-28. (canceled)

29. A method of controlling a nested pair of rigidizing devices, the method comprising:
receiving a copy command from a user input;
automatically performing a shape copying sequence, wherein the shape copying sequence comprises:
advancing a first rigidizing device of the nested pair of rigidizing devices relative to a second rigidizing device of the nested pair of rigidizing devices, while the first rigidizing device is in a flexible state and the second rigidizing device is in a rigidized state, wherein the first rigidizing device is initially proximal to the second rigidizing device so that first rigidizing device copies the shape of the second rigidizing device; and
preventing the first rigidizing device from advancing distal to the second rigidizing device.

30. The method of claim 29, wherein advancing the first rigidizing device comprises advancing the first rigidizing device only while the copy command is continuously received.

31. The method of claim 29, wherein further comprising continuing advancement of the first rigidizing device until a distal end of the first rigidizing device reaches a distal end of the second rigidizing device.

32. The method of claim 29, wherein the first rigidizing device is nested over the second rigidizing device.

33. The method of claim 29, wherein the shape copying sequence further comprises comprising rigidizing the second rigidizing device into the rigid state prior to advancing the first rigidizing device.

34. The method of claim 29, wherein the shape copying sequence further comprises de-rigidizing the first rigidizing device into the flexible state prior to advancing the first rigidizing device relative to the second rigidizing device.

35. The method of claim 29, wherein the shape copying sequence further comprises rigidizing the first rigidizing device into the rigid state after it has advanced relative to the second rigidizing device.

36. The method of claim 29, further comprising, prior to receiving the copy command, advancing the second rigidizing device in the flexible state while steering a distal end region of the second rigidizing device, wherein the first rigidizing device is in the rigid state.

37. A method of controlling a nested pair of rigidizing devices, the method comprising:
receiving a copy command from a user input; and
automatically performing a shape copying sequence while the user input is received, wherein the shape copying sequence comprises:
advancing a first rigidizing device of the nested pair of rigidizing devices relative to the first to a second rigidizing device of the nested pair of rigidizing devices, while the first rigidizing device is in a flexible state and the second rigidizing device is in a rigidized state, wherein the first rigidizing device is initially proximal to the second rigidizing device so that first rigidizing device copies the shape of the second rigidizing device.

38. A system comprising:
a nested pair of rigidizing devices comprising a first rigidizing device and a second rigidizing device;
one or more processors; and
a memory coupled to the one or more processors, the memory storing computer-program instructions, that, when executed by the one or more processors, perform a computer-implemented method for controlling the nested pair of rigidizing devices, the method comprising:
receiving a copy command from a user input;
automatically performing a shape copying sequence, wherein the shape copying sequence comprises:
advancing a first rigidizing device relative to a second rigidizing device, while the first rigidizing device is in a flexible state and the second rigidizing device is in a rigidized state, wherein the first rigidizing device is initially proximal to the second rigidizing device so that first rigidizing device copies the shape of the second rigidizing device; and
preventing the first rigidizing device from advancing distal to the second rigidizing device.

39. The system of claim 38, wherein advancing the first rigidizing device comprises advancing the first rigidizing device only while the copy command is continuously received.

40. The system of claim 38, wherein the computer-implemented method further comprises continuing advancement of the first rigidizing device until a distal end of the first rigidizing device reaches a distal end of the second rigidizing device.

41. The system of claim 38, wherein the first rigidizing device is nested over the second rigidizing device.

42. The system of claim 38, wherein the shape copying sequence further comprises comprising rigidizing the second rigidizing device into the rigid state prior to advancing the first rigidizing device.

43. The system of claim 38, wherein the shape copying sequence further comprises de-rigidizing the first rigidizing device into the flexible state prior to advancing the first rigidizing device relative to the second rigidizing device.

44. The system of claim 38, wherein the shape copying sequence further comprises rigidizing the first rigidizing device into the rigid state after it has advanced relative to the second rigidizing device.

45. The system of claim 38, wherein the computer-implemented method further comprises, prior to receiving the copy command, advancing the second rigidizing device in the flexible state while steering a distal end region of the second rigidizing device, wherein the first rigidizing device is in the rigid state.

46. A system comprising:
a nested pair of rigidizing devices comprising a first rigidizing device and a second rigidizing device;
one or more processors; and
a memory coupled to the one or more processors, the memory storing computer-program instructions, that, when executed by the one or more processors, perform a computer-implemented method for controlling the nested pair of rigidizing devices, the method comprising:
receiving a copy command from a user input;
automatically performing a shape copying sequence while the copy command is received, wherein the shape copying sequence comprises:
advancing a first rigidizing device relative to a second rigidizing device, while the first rigidizing device is in a flexible state and the second rigidizing device is in a rigidized state, wherein the first rigidizing device is initially proximal to the second rigidizing device so that first rigidizing device copies the shape of the second rigidizing device.

47.-135. (canceled)
